SEQUENCE LISTING (42 sequences)

SEQ ID NO: 1 - DNA, 22 bp, Glycine max
aaggtctctgtcttaatggtga

SEQ ID NO: 2 - DNA, 22 bp, Glycine max
tcttgtccaaaccatagtccaa

SEQ ID NO: 3 - DNA, 22 bp, Glycine max
atgggtggaatagggaaaacaa

SEQ ID NO: 4 - DNA, 22 bp, Glycine max
caaccttgatttccttgattaa

SEQ ID NO: 5 - DNA, 22 bp, Glycine max
caatgcctattttagaaatgaa

SEQ ID NO: 6 - DNA, 22 bp, Glycine max
aggtggaataggaaaaacaact

SEQ ID NO: 7 - DNA, 22 bp, Glycine max
tcaccattaagacagagacctt

SEQ ID NO: 8 - DNA, 22 bp, Glycine max
taggactatggtttggacaagt

SEQ ID NO: 9 - DNA, 22 bp, Glycine max
ttgttttacctattccacccat

SEQ ID NO: 10 - DNA, 22 bp, Glycine max
ttaatcaaggaaatcacggtcg

SEQ ID NO: 11 - DNA, 22 bp, Glycine max
ttaatcaaggaaatcacggttg

SEQ ID NO: 12 - DNA, 22 bp, Glycine max
ttcatttttaaaataggcattg

SEQ ID NO: 13 - DNA, 22 bp, Glycine max
tgttgttttacctattccacct

SEQ ID NO: 14 - DNA, 21 bp, Glycine max
ttcatttttaaaataggcatt

SEQ ID NO: 15 - DNA, 24 bp, Artificial Sequence
  Synthetic Oligonucleotide Primer
gtcttgatgcacagttgctgcagc

SEQ ID NO: 16 - DNA, 26 bp, Artificial Sequence
  Artificial Oligonucleotide Primer
ctcgggtacaatggaacatagtctcc

SEQ ID NO: 17 - DNA, 22 bp, Artificial Sequence
  Artificial Oligonucleotide Primer
gccgacaagcagaagaacggca

SEQ ID NO: 18 - DNA, 22 bp, Artificial Sequence
  Artificial Oligonucleotide Primer
aggtagtggttgtcgggcagca

SEQ ID NO: 19 - DNA, 24 bp, Artificial Sequence
  Artificial Oligonucleotide Primer
acagaagctgaagtgactgggctt

SEQ ID NO: 20 - DNA, 24 bp, Artificial Sequence
  Artificial Oligonucleotide Primer
cacagctgaaaggtactggttgtt

SEQ ID NO: 21 - DNA, 29 bp, Artificial Sequence
  Artificial Oligonucleotide Primer
gttcgagtattatggcattgggaaaactg

SEQ ID NO: 22 - DNA, 25 bp, Artificial Sequence
  Artificial Oligonucleotide Primer
cacagttcgatagcgaaaaccgaat

SEQ ID NO: 23 - DNA, 54 bp, Artificial Sequence
  Artificial Oligonucleotise Primer
atataggcgcgcccaatgcctattttagaaatgaacaggtcttgctaggggtgc

SEQ ID NO: 24 - DNA, 33 bp, Artificial Sequence
  Artificial Oligonucleotide Primer
tatatcctaggctcgcctctcagttctgcataa

SEQ ID NO: 25 - DNA, 58 bp, Artificial Sequence
  Artificial Oligonucleotide Primer
atataggcgcgcccaatgcctattttagaaatgaacttagggccaacaagctcaaggg

SEQ ID NO: 26 - DNA, 33 bp, Artificial Sequence
  Artificial Oligonucleotide Primer
tatatcctaggctgatttgcttgcacttttgcg

SEQ ID NO: 27 - DNA, 32 bp, Artificial Sequence
  Artificial Oligonucleotide Primer
atataggcgcgcccaggtcttgctaggggtgc

SEQ ID NO: 28 - DNA, 33 bp, Artificial Sequence; identical to SEQ ID NO: 24
  Artificial Oligonucleotide Primer

SEQ ID NO: 29 - DNA, 36 bp, Artificial Sequence
  Artificial Oligonucleotide Primer
atataggcgcgcccttagggccaacaagctcaaggg

SEQ ID NO: 30 - DNA, 33 bp, Artificial Sequence; identical to SEQ ID NO: 26
  Artificial Oligonucleotide Primer

SEQ ID NO: 31 - DNA, 51 bp, Artificial Sequence
  Artificial Oligonucleotide Primer
atataggcgcgcccaaccttgatttccttgattaactcgtgaccaccttca

SEQ ID NO: 32 - DNA, 29 bp, Artificial Sequence
  Artificial Oligonucleotide Primer
tatatcctagggccgttcttctgcttgtc

SEQ ID NO: 33 - DNA, 51 bp, Artificial Sequence
  Artificial Oligonucleotide Primer
atataggcgcgccaggtggaataggaaaaacaactctcgtgaccaccttca

SEQ ID NO: 34 - DNA, 51 bp, Artificial Sequence
  Artificial Oligonucleotide Primer
atataggcgcgccatgggtggaatagggaaaacaactcgtgaccaccttca

SEQ ID NO: 35 - DNA, 51 bp, Artificial Sequence
  Artificial Oligonucleotide Primer
atataggcgcgcccaatgcctattttagaaatgaactcgtgaccaccttca

SEQ ID NO: 36 - DNA, 51 bp, Artificial Sequence
  Artificial Oligonucleotide Primer
atataggcgcgccaaggtctctgtcttaatggtgactcgtgaccaccttca

SEQ ID NO: 37 - DNA, 51 bp, Artificial Sequence
  Artificial Oligonucleotide Primer
atataggcgcgcctcttgtccaaaccatagtccaactcgtgaccaccttca

SEQ ID NO: 38 - DNA, 29 bp, Artificial Sequence
  Artificial Oligonucleotide Primer
atataggcgcgccctcgtgaccaccttca

SEQ ID NO: 39 - DNA, 60 bp, Artificial Sequence
  Artificial Oligonucleotide Primer
ggccgcaccggcgcgcccaatgcctattttagaaatgaaccatcaagacgttctccgtga

SEQ ID NO: 40 - DNA, 37 bp, Artificial Sequence
  Artificial Oligonucleotide Primer
aacctaggaaggatccactttggccgcttcgagatca

SEQ ID NO: 41 - DNA, 21 bp, Artificial Sequence
  Artificial Oligonucleotide Primer
cgagattgcttcagatccgta

SEQ ID NO: 42 - DNA, 20 bp, Artificial Sequence
  Artificial Oligonucleotide Primer
cggtcattagaggccacgat

(19) United States
(12) Patent Application Publication
(10) Pub. No.: US 2015/0089688 A1
(43) Pub. Date: Mar. 26, 2015

(54) COMPOSITIONS AND METHODS OF GENE SILENCING IN PLANTS

(71) Applicants: University of Georgia Research Foundation, Inc., Athens, GA (US); The Board of Trustees of the University of Illinois, Urbana, IL (US)

(72) Inventors: Thomas B. Jacobs, Athens, GA (US); Wayne A. Parrott, Athens, GA (US); Lila O. Vodkin, Champaign, IL (US)

(21) Appl. No.: 14/559,209

(22) Filed: Dec. 3, 2014

Related U.S. Application Data
(63) Continuation of application No. PCT/US2013/044267, filed on Jun. 5, 2013.
(60) Provisional application No. 61/655,810, filed on Jun. 5, 2012.

(51) Int. Cl. C12N 15/82

(52) U.S. Cl. CPC C12N 15/8218; C12N 15/8241; C12N 15/8271; C12N 15/8279; C12N 15/8247; C12N 15/8245; C12N 15/8249; C12N 15/825; C12N 15/8255; C12N 15/8243; C12N 15/8289; C12N 15/8251
USPC 800/279; 435/320.1; 800/298; 800/312; 800/286; 800/281; 800/282; 800/283; 800/284; 800/290

(57) ABSTRACT

Compositions and methods for inducing gene silencing events in plants are disclosed. The compositions typical include a polynucleotide encoding an miRNA target sequence operably linked to a sequence of from a target gene, cDNA or mRNA, or fragment thereof. When expressed in the presence of an miRNA specific for the miRNA target sequence the compositions can induce production of trans-acting siRNA that silence the target of interest. Transgenic plants and preferred plant pathways that can be targeted using the disclosed methods and compositions are also disclosed.

### CROSS-REFERENCE TO RELATED APPLICATION

This application is a continuation of International Application No. PCT/US2013/044267 filed under the Patent Cooperation Treaty on Jun. 5, 2013, which claims benefit of and priority to U.S. Provisional Patent Application No. 61/655,810 filed Jun. 5, 2012, all of which are incorporated herein by reference in their entirety.

### REFERENCE TO SEQUENCE LISTING

The Sequence Listing submitted on Dec. 3, 2014 as a text file named “UGA_{—}1920_PCT_ST25_txt,” created on Jun. 5, 2013, and having a size of 8,192 bytes is hereby incorporated by reference pursuant to 37 C.F.R. §1.52(e)(5).

### FIELD OF THE INVENTION

The invention is generally related to compositions and methods for gene silencing in plants using the trans-acting siRNA pathway.

### BACKGROUND OF THE INVENTION

Gene silencing plays a significant role in crop trait development. Gene expression relies on the transcription of DNA into mRNA and the translation of mRNA into protein. Gene silencing limits the amount of mRNA available for translation and reduces the amount of protein produced. There are many agronomical traits that can be attributed to gene silencing. Buff versus black seed coats of soybean (Tuteja, et al., Plant Cell 21:3063-3077 (2009)) and some types of red-stemmed versus green-stemmed corn (Della Vedova, et al., Genetics 170:1989-2002 (2005)). The Flavr Savr tomato, which was the first transgenic plant released in 1994, was developed using the gene silencing pathways. Although the exact mechanism was unknown at the time, it is now known that it was derived from the activation of the gene silencing pathways Krieger, et al., Hortscience 43:962-964 (2008)). Since the release of the Flavr Savr tomato, gene silencing strategies have been used to produced insect resistant corn (Baum, et al., Nature Biotechnology 25:1322-1326. (2007)) and cotton (Mao, et al., Nature Biotechnology 25:1307-1313 (2007)), powdery mildew resistance in wheat and barley (Nowara, et al., Plant Cell 22:3130-3141 (2010)), nematode resistance in arabidopsis (Huang, et al., Proceedings of the National Academy of Sciences of the United States of America 103:14302-14306 (2006)) and soybean (Klink, et al., Planta 230:53-71 (2009)) and high oleic varieties of soybean (Flores, et al., Transgenic Research 17:839-850 (2008)). Although gene silencing has demonstrated feasibility in the development of commercially valuable transgenic plants, use of the prevailing technology is limited by a number of factors.

One mechanism of gene silencing is through the production of small RNA (sRNA) complementary to a gene target. These sRNAs are incorporated into an RNA-induced silencing complex (RISC) and specifically target transcripts with near perfect homology. A sRNA can be derived from various sources: hairpin vectors, viral RNA, transposon intermediates, and endogenous loci. In plants, dsRNAs are processed into 20-25-nt RNA molecules by Dicer-like (DCL) enzymes.

Micro-RNAs (miRNAs) are produced from transcripts that form hairpins upon transcription. The hairpins are typically composed of imperfectly complementary sequences. From a miRNA transcript, a single 21 nucleotide (nt) species of sRNA can be released and incorporated into a RISC that specifically cleaves homologous mRNA sequences. The cleaved sequences are then degraded by normal cellular processes. Endogenous miRNAs are important for several cellular processes, including development and response to stress (Voinnet, Cell 136:669-687 (2009)).

Small-interfering RNAs (siRNA) are produced from RNA hairpins derived from long inverted repeats, viral replication, and hairpin vectors. The hairpins are cleaved to release a large variety of sRNA species 21- and 22-nt in length. The hairpins are typically composed of perfectly complementary sequences. These sRNAs are incorporated into a RISC complex and cleave homologous transcripts (Brodersen, et al., Trends in Genetics 22:268-280. (2006). The cleaved fragments are not always degraded as in the miRNA pathway. Instead the fragments can serve as a template for an RNA-dependent RNA-polymerase (RDR) that will synthesize a dsRNA molecule that can lead to the production of secondary siRNAs (Voinnet, Trends in Plant Science 13:317-328 (2008)). This process, termed transitivity, can result in the production of siRNA species different than those produced from the initial dsRNA and induce the silencing of other closely related genes. Because of transitivity, the potential for off-target effects is always a concern when using hairpin vectors.

Current strategies to induce gene silencing in plants rely on the use of hairpin vectors (Wesley, et al., Plant Journal 27:581-590 (2001)). They are so named because the DNA sequence is arranged as an inverted-repeat so that upon transcription, the mRNA molecule will fold back to form a hairpin structure. These vectors are effective at generating silenced events; however there are some difficulties using them. The construction of hairpin vectors is complex due to the inverted-repeat, and there appears to be some instability in the common bacterial strains used. Furthermore, the incorporation of the DNA into the plant genome also appears to be unstable as events can be produced that lack either one of the inverted regions (Sunitha, et al., Plant Molecular Biology Reporter 30:158-167 (2012)). Of the events that do contain the complete inverted-repeat, only half of the events induce silencing of the target gene while the other half are unaffected. All of these complications makes the generation of silenced events time-consuming and expensive. Currently there are no other widely adopted alternatives for the generation of silenced events in plants.

Therefore, it is an object of the invention to provide alternative compositions and methods for gene silencing in plants.

### SUMMARY OF THE INVENTION

Compositions and methods for inducing gene silencing events in plants are disclosed. The compositions typically include a polynucleotide encoding an miRNA target sequence operably linked to a sequence from a target gene, cDNA or mRNA, or fragment thereof. In some embodiments, the two sequences are directly linked. The compositions can be RNA, DNA or a combination thereof.

When transfected into a plant cell expressing an miRNA specific for the miRNA target sequence, the compositions can be used to induce production of trans-acting small-interfering RNAs (tasiRNA) that mediate silencing of the target gene. A sequence encoding the miRNA can be operably linked to the polynucleotide encoding the miRNA target sequence and the target gene sequence, or expressed separately. In one embodiment, the miRNA target sequence is an endogenous soybean miRNA target sequence.

The compositions and methods can be used to generate transgenic plants, for example transgenic soybean plants. In some embodiments the transgenic plants are pathogen or pest-resistant, for example nematode resistant.

For example, recombinant nucleic acid constructs including a tasiRNA-inducing miRNA recognition sequence and a coding region of a target gene are disclosed. The miRNA recognition sequence can include the sequence of SEQ ID NOs 1, 2, 3, 4, 5, or 6. The coding region of the target gene can be, for example, an exon of the target gene or a fragment thereof.

The recombinant nucleic acid construct can include a terminator. The recombinant nucleic acid construct can include a promoter. The nucleic acid can be incorporated into an expression vector, such as a vector suitable for transforming the nucleic acid construct into a plant genome. The plant genome can be a nuclear, mitochondrial, or plastid genome. The constructs and vectors can be used to create transgenic plants with reduced gene expression.

For example, a method of reducing or inhibiting gene expression in a plant cell can include transforming the plant cell with a vector that expresses a recombinant nucleic acid construct including a tasiRNA-inducing miRNA recognition sequence, such as SEQ ID NOs 1, 2, 3, 4, 5, or 6 and a coding region of a target gene. Co-expression of an miRNA that binds to the miRNA target sequence induces production of trans-acting siRNA in the cell. The coding region of the target gene can be 10, 20, 30, 50, 75, 100, 1250, 150, 200, 250, 500, 750, 1000, or more nucleotides. In some embodiments, the sequence encoding the miRNA is incorporated into the genome of the cell. In some embodiments, the sequence encoding the miRNA is expressed extra-chromosomally. The miRNA sequence is heterologous or endogenous.

The method can be used to reduce or inhibit expression of a target gene that affects a pathway in the plant, for example, a pathway related to resistance to pathogens, resistance to abiotic stress factors, a biosynthetic pathway, modification of the composition or content of fatty acids, lipids or oils modification of the carbohydrate composition, modification of color or pigmentation, reduction of storage protein content, prevention of stem break, delay of fruit maturation, induction of male sterility, reduction of undesired or toxic plant constituents, delay of senescence symptoms, modification of the lignification or the lignin, or modification of fiber content or quality.

The methods can also be used to reduce or inhibit gene expression in a plant. Transgeneic plants transformed with a recombinant nucleic acid construct including a tasiRNA-inducing miRNA recognition sequence, such as SEQ ID NOs 5, 1, 2, 3, 4 or 6 and a coding region of a target gene are also disclosed. The transgenic plant can also include a polynucleotide encoding an miRNA that binds under stringent conditions to the miRNA target sequence. The miRNA can have, for example, a sequence at least 90% identical to SEQ ID NO: 12, 7, 8, 9, 10, 11, 13 or a complement thereof.

In some embodiments, the plant is a soybean plant.

### BRIEF DESCRIPTION OF THE DRAWINGS

FIG. 1A is a diagram illustrating the mechanism of tasiRNA. A RISC complex, guided by a miRNA, induces the cleavage of a mRNA with a miRNA recognition sequence. In the tasiRNA pathway, RDR6 synthesizes a dsRNA from the 3′ cleavage product. DCL4 then cleaves the newly formed dsRNA to produce siRNAs that can silence complementary mRNAs. FIG. 1B is a schematic of a generic tasiRNA vector construct including a promoter driving expression of a (22-nucleotide) miRNA recognition sequence operably linked to a target sequence of interest and a terminator. By fusing a tasiRNA-inducing miRNA recognition sequence to a either a whole or partial gene of interest, siRNAs will be made from that sequence and induce silencing of complementary transcripts. FIG. 1C is a schematic of an exemplary tasiRNA vector construct including a GmUbi promoter driving expression of an miR1514 recognition sequence operably linked a target sequence of interest and a rbcS Terminator. FIG. 1D is a flow chart illustrating how tasiRNA silencing vectors induce gene silencing.

FIG. 2 is a bar graph showing expression patterns of the putative tasiRNA-inducing miRNAs 3514, 1509b.2, 1510a.2, 1510, 1514a.2, and 5770.2 (normalized counts per million reads) in various tissue types.

FIGS. 3A and 3B are dot plots showing mRNA expression levels (relative to empty vector control) for gene-silencing vectors made using the 1514a.2 recognition sequence fused to 359 bp and 346 bp portions of P450 (3A) and NFR (3B) and introduced into soybean via hairy-root transformation (referred to as 1514:P450 and 1514:NFR); control vectors without the miRNA recognition sequence (referred to as P450 and 1514:NFR); and empty-vector (wild-type A. rhizogenes).

FIGS. 4A and 4B are each a series of coverage graphs of assembled sRNA reading to vector and mRNA sequences from P450 (4A) and NFR (4B). Sequences that assembled in the sense orientation are in the +y axis and sequences that assembled in the reverse complement orientation are in the −y axis. Scales are normalized per millions of counts.

FIG. 5 is a dot plot showing mRNA expression levels and GFP protein abundance (relative to empty vector controls) from ten randomly selected sGFP gene-silencing events in transgenic soybean line homozygous for sGFP transformed with a gene-silencing vector made for sGFP with one of six putative miRNA recognition sequences (1509, 1510, 1510a.2, 1514a.2, 3514, and 5770.2).

### DETAILED DESCRIPTION OF THE INVENTION

### I. Definitions

Unless otherwise indicated, the disclosure encompasses conventional techniques of plant breeding, microbiology, cell biology and recombinant DNA, which are within the skill of the art. See, e.g., Sambrook and Russell, Molecular Cloning: A Laboratory Manual, 3rd edition (2001); Current Protocols In Molecular Biology [(F. M. Ausubel, et al. eds., (1987)]; Plant Breeding: Principles and Prospects (Plant Breeding, Vol 1) M. D. Hayward, N. O. Bosemark, I. Romagosa; Chapman & Hall, (1993); Coligan, Dunn, Ploegh, Speicher and Wingfeld, eds. (1995) Current Protocols in Protein Science (John Wiley & Sons, Inc.); the series Methods in Enzymology (Academic Press, Inc.): PCR 2: A Practical Approach (M. J. MacPherson, B. D. Hames and G. R. Taylor eds. (1995)].

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience., 1999; and Robert A. Meyers (ed.), Molecular Biology and Biotechnology, a Comprehensive Desk Reference, published by VCH Publishers, Inc., 1995; Sambrook and Russell. (2001) Molecular Cloning: A Laboratory Manual 3rd. edition, Cold Spring Harbor Laboratory Press.

To facilitate understanding of the disclosure, the following definitions are provided:

The term “trans-acting small interfering RNA,” “trans-acting siRNA,” and “tasiRNA” are used interchangeable and refer to a specific loci that target mRNA sequences in trans. Trans-acting siRNAs (tasiRNAs) negatively regulate target transcripts and are characterized by siRNAs spaced in about 21-nucleotide (nt) “phased” intervals, a pattern thought to be formed by DICER-LIKE 4 (DCL4) processing. The production of the siRNAs is initiated by a miRNA recognition sequence, also referred to as a miRNA target sequence, cleaved by a miRNA.

The terms “phased small interfering RNA,” “phased siRNA,” and “phasiRNA,” as used interchangeably and describe loci that produce siRNA in a phased pattern, but have not met all criteria to be called tasiRNA.

The terms “microRNA” and “miRNA” are used interchangeably and refer to a short ribonucleic acid (RNA) molecule which can bind by complementary base pairing to a target messenger RNA transcripts (mRNAs), usually causing translational repression or target degradation which results in gene silencing.

The terms “miRNA recognition sequence” and “miRNA target sequence” are used interchangeably and refer to a sequence that can hybridize with an miRNA and induce the tasiRNA pathway.

The term “plant” is used in its broadest sense. It includes, but is not limited to, any species of woody, ornamental or decorative crop or cereal, and fruit or vegetable plant. It also refers to a plurality of plant cells that are largely differentiated into a structure that is present at any stage of a plant's development. Such structures include, but are not limited to, a fruit, shoot, stem, leaf, flower petal, etc.

The term “non-naturally occurring plant” refers to a plant that does not occur in nature without human intervention. Non-naturally occurring plants include transgenic plants and plants produced by non-transgenic means such as plant breeding.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

The term “plant material” refers to leaves, stems, roots, flowers or flower parts, fruits, pollen, egg cells, zygotes, seeds, cuttings, cell or tissue cultures, or any other part or product of a plant. The term “plant organ” refers to a distinct and visibly structured and differentiated part of a plant such as a root, stem, leaf, flower bud, or embryo.

The term “plant cell” refers to a structural and physiological unit of a plant, comprising a protoplast and a cell wall. The plant cell may be in form of an isolated single cell or a cultured cell, or as a part of higher organized unit such as, for example, a plant tissue, a plant organ, or a whole plant.

The term “plant cell culture” refers to cultures of plant units such as, for example, protoplasts, cell culture cells, cells in plant tissues, pollen, pollen tubes, ovules, embryo sacs, zygotes and embryos at various stages of development.

The term “transgenic plant” refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

The term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term “orthologous genes” or “orthologs” refer to genes that have a similar nucleic acid sequence because they were separated by a speciation event.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids are at least 60% free, preferably 75% free, and most preferably 90% free from other associated components. An “isolated” nucleic acid molecule or polynucleotide is a nucleic acid molecule that is identified and separated from at least one contaminant nucleic acid molecule with which it is ordinarily associated in the natural source. The isolated nucleic can be, for example, free of association with all components with which it is naturally associated. An isolated nucleic acid molecule is other than in the form or setting in which it is found in nature.

As used herein, the term “locus” refers to a specific position along a chromosome or DNA sequence. Depending upon context, a locus could be a gene, a marker, a chromosomal band or a specific sequence of one or more nucleotides.

The term “vector” refers to a replicon, such as a plasmid, phage, or cosmid, into which another DNA segment may be inserted so as to bring about the replication of the inserted segment. The vectors can be expression vectors.

The term “expression vector” refers to a vector that includes one or more expression control sequences.

The term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

The term “promoter” refers to a regulatory nucleic acid sequence, typically located upstream (5′) of a gene or protein coding sequence that, in conjunction with various elements, is responsible for regulating the expression of the gene or protein coding sequence. The promoters suitable for use in the constructs of this disclosure are functional in plants and in host organisms used for expressing the disclosed polynucleotides. Many plant promoters are publicly known. These include constitutive promoters, inducible promoters, tissue- and cell-specific promoters and developmentally-regulated promoters. Exemplary promoters and fusion promoters are described, e.g., in U.S. Pat. No. 6,717,034, which is herein incorporated by reference in its entirety.

A nucleic acid sequence or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking can be accomplished by ligation at convenient restriction sites. If such sites do not exist, synthetic oligonucleotide adaptors or linkers are used in accordance with conventional practice.

“Transformed,” “transgenic,” “transfected” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

The term “endogenous” with regard to a nucleic acid refers to nucleic acids normally present in the host.

The term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompasses “exogenous” and “non-native” elements.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 [symbol] g/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Third Edition, Cold Spring Harbor, N.Y. (2000).

### II. Compositions for Generating Trans-Acting Small-Interfering RNAs

Compositions and methods for inducing gene silencing events in plants are disclosed. The compositions disclosed herein typically include a polynucleotide encoding an miRNA target sequence operably linked to a sequence from a gene or mRNA of interest, and optionally a miRNA sequence. When the polynucleotide encoding an miRNA target sequence operably linked to a sequence from a gene or mRNA of interest is expressed in combination with a miRNA specific for the miRNA target sequence in cell, the cell produces tasiRNA directed against the gene or mRNA of interest. As discussed in more detail below, the miRNA target sequence operably linked to a sequence from a gene or mRNA of interest, and optional miRNA sequences are generally incorporated into one or more expression vectors suitable for expression in a cell of interest, such as a plant cell. The sequences can endogenous or heterologous.

A. miRNA-Recognition Sequence-Target Gene Constructs

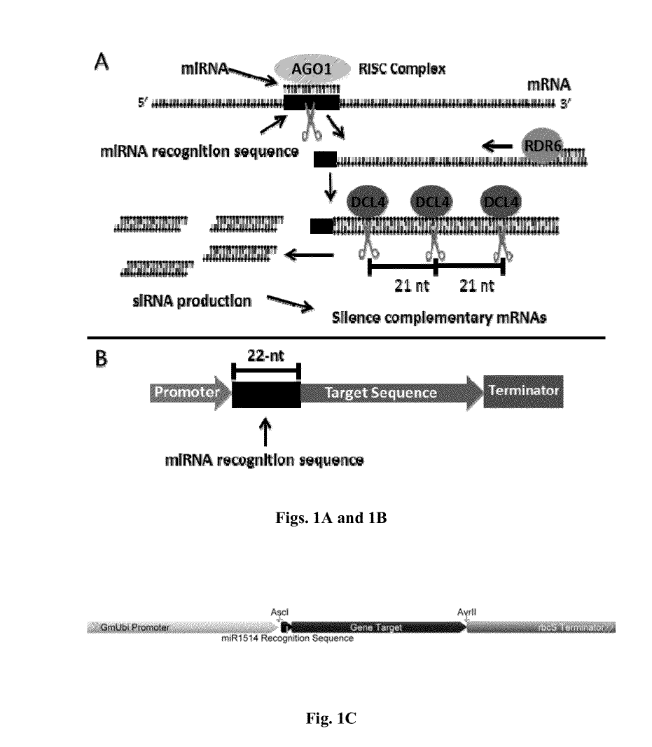

Trans-acting small-interfering RNAs (tasiRNA) are siRNAs that are produced by cleavage of an endogenous single-stranded transcript by a miRNA (FIGS. 1A-1D). Rather than the cleaved transcript being degraded, RDR6 synthesizes a dsRNA molecule from the 3′ cleavage fragment. Since the initial cleavage site is determined by a miRNA, an identical dsRNA molecule is formed each time. DCL4 then processes the dsRNA from the cleaved end, resulting in siRNAs being released in a phased pattern, every 21-nt from the initial cleavage site. Just as in the siRNA pathway, tasiRNAs can be incorporated into a RISC and specifically cleave homologous transcripts (Yoshikawa, Genes & Development 19:2164-2175 (2005)). The tasiRNA pathway has been used to generate silenced transgenic plants (Montgomery, et al., Proceedings of the National Academy of Sciences of the United States of America 105:20055-20062 (2008)). Insertion of the target for the arabidopsis miRNA, miR173, into a phytoene desaturase gene (PDS) resulted in silencing in trans of the native PDS gene, as evidenced by photobleaching and a reduction in PDS mRNA levels. The tasiRNAs were only produced 3′ to the target site and were dependent on RDR6 and DCL4. Thus far several bona-fide and putative-tasiRNA loci have been identified in Arabidopsis (Manavella, et al., Proceedings of the National Academy of Sciences of the United States of America 109:2461-2466 (2012)) and Medicago truncatula (Zhai, et al., Genes & Development 25:2540-2553. (2011)) and other species (reviewed in (Allen, et al., Seminars in Cell & Developmental Biology 21:798-804. (2010)). In this system a single ˜22-nt miRNA target site is sufficient to produce siRNA to a specific sequence.

In order to use the tasiRNA pathway to induce gene-silencing in plants, the miRNA recognition sequences must be known. Soybean miRNA-recognition sequences for generating phasiRNA are disclosed.

1. miRNA Target Sequences

Isolated miRNA target sequences are disclosed. An miRNA target or recognition sequence is a sequence, or the complement thereof, that can be hybridized by an miRNA. In some embodiments, the miRNA target sequence is perfectly complementary to the sequence an miRNA. In some embodiments, the miRNA target sequence is not perfectly complementary to the miRNA, but hybridizes to the miRNA under stringent conditions.

The miRNA target sequence typically has a length similar to the length of the natural microRNA binding site, which are known in the art to typically include between about 15 and 30 nucleotides, preferably, about 20 to about 28 nucleotides, more preferably about 21-24 nucleotides. In some embodiments, the sequence has the same length than the microRNA binding site, for example 20, 21, 22, 23, or 24 nucleotides. In some embodiments the miRNA target sequence is 22 nucleotides in length. In some embodiments, the miRNA target sequence is longer or shorter than microRNA binding sites, for example by one, two, three, or more nucleotides).

The miRNA target sequence can be naturally occurring or a non-naturally occurring sequence. In some embodiments, the miRNA target sequence is a sequence that occurs naturally in a plant. For example, the miRNA target sequence can be an miRNA target sequence endogenous to soybean. Example of isolated miRNA target sequences disclosed herein include:

(SEQ ID NO: 1)
AAGGTCTCTGTCTTAATGGTGA,
(SEQ ID NO: 2)
TCTTGTCCAAACCATAGTCCAA,
(SEQ ID NO: 3)
ATGGGTGGAATAGGGAAAACAA,
(SEQ ID NO: 4)
CAACCTTGATTTCCTTGATTAA,
(SEQ ID NO: 5)
CAATGCCTATTTTAGAAATGAA,
and
(SEQ ID NO: 6)
AGGTGGAATAGGAAAAACAACT.

A polynucleotide having the nucleic acid sequence SEQ ID NO: 1, 2, 3, 4, 5, 6, and complements and combinations thereof are therefore disclosed. A polynucleotide can have a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1, 2, 3, 4, 5, 6, 7, or a complement or combination thereof. A polynucleotide that hybridizes under stringent conditions to a polynucleotide consisting of the nucleic acid sequence SEQ ID NO: 1, 2, 3, 4, 5, 6 and complements and combinations thereof are also disclosed.

A polynucleotide that is a fragment of SEQ ID NO: 1, 2, 3, 4, 5, 6, or complement, or combination thereof are also disclosed. Therefore, a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of SEQ ID NO: 1, 2, 3, 4, 5, 6, and complements and combinations thereof is disclosed. The fragment can be at least 1, 2, 3, 4, 5, or more nucleotides shorter than SEQ ID NO: 1, 2, 3, 4, 5, or 6.

Other miRNA target sequences suitable for use in the disclosed compositions and methods are known in the art, see for example, miRBase, release 18.

2. Target of Interest

The miRNA target sequences disclosed herein can be operably linked a target gene, cDNA, mRNA, or other nucleotide of interest to induce the tsaiRNA pathway and production of silencing RNA against the target gene, cDNA, mRNA, or other nucleotide of interest. Therefore compositions disclosed herein can include a sequence encoding a gene, cDNA or mRNA of interest, or fragment thereof. The sequence can be 70%, 75%, 80%, 85%, 90%, 95%, 98%, 99%, or 100% identical to the sequence of the endogenous target gene, cDNA or mRNA, or 70%, 75%, 80%, 85%, 90%, 95%, 98%, 99%, or 100% identical to a complement thereof.

The polynucleotide of interest is operably linked to the polynucleotide encoding an miRNA target sequence. The miRNA target sequence can be linked or fused directly to the sequence encoding the gene, cDNA or mRNA of interest, or it can be linked indirectly. In some embodiments there is one or more linking nucleotides between the miRNA target sequence and the sequence encoding the gene or mRNA of interest. The two sequences should be linked such that when the miRNA target sequence is bound by an miRNA, the resulting miRNA target sequence-miRNA hybrid nucleotide directs biogenesis of tasiRNA specific for the gene or mRNA of interest.

The polynucleotide of interest can be a full-length gene, or complement thereof. It can include non-coding regions including, but not limited to 5′ untranslated region, 3′ untranslated region, and one or more introns. The polynucleotide of interest can be a gene's coding region, or complement thereof, for example an mRNA or cDNA.

The polynucleotide can be a fragment of a full-length gene, mRNA, or cDNA. The polynucleotide can include the coding region, one or more introns, 5′ untranslated region, 3′ untranslated region or a combination thereof from a full-length gene. For example, the polynucleotide can at least 10, preferably at least 20, more preferably at least 30, most preferably at least 50 nucleotides of a gene, mRNA, or cDNA of interest. In some embodiments, the polynucleotide includes the first 10, 20, 30, 40, 50, 75, 100, 150, 200, 300, 400, 500, 750, or 1000 nucleotides of a gene or mRNA numbering for the 5′ ATG start site. In some embodiments, the polynucleotide includes 10, 20, 30, 40, 50, 75, 100, 150, 200, 300, 400, 500, 750, or 1000 nucleotides beginning 3′ of the ATG start site. In some embodiments, the polynucleotide includes the last 10, 20, 30, 40, 50, 75, 100, 150, 200, 300, 400, 500, 750, or 1000 nucleotides ending with the 3′ stop codon. In some embodiments the polynucleotide includes the entire transcriptional unit of the gene, mRNA, or cDNA of interest.

In some embodiments the polynucleotide directs formation of tasiRNA against all splice variants of a gene or mRNA of interest by including sequences that are common to all of the splice variants. Likewise, the polynucleotide can direct formation of tasiRNA against related genes or mRNA of interest by including one or more sequences that are similar or related between the two related genes. For example, in some embodiments, the polynucleotide includes a sequence that 80%, 85%, 90%, 95%, 98%, 99%, or 100% identical to a sequence found in at least two different genes or mRNA of interest. The polynucleotide can also be specific for one or more splice variants of a gene when the sequence of the polynucleotide is unique that one or more splice variants.

The possible target genes of the compositions disclosed herein include but are not limited to those discussed below. The genes are generally related to one or more functions or pathways in a cell. It is also possible to target a single gene or mRNA. It is also possible to target more than one gene or mRNA simultaneously. Therefore, in some embodiments, the expression of at least two different target genes is reduced. The target genes can originate from a single group of genes direct to the same or related function or pathway. Alternatively, target genes can originate from genes directed to different or unrelated functions or pathways.

B. miRNA

miRNA target single-stranded non-coding RNA transcripts, such as the miRNA target sequence discussed above, and direct biogenesis of tasiRNA via initiation of a double-stranded RNA produced by RNA-dependent RNA polymerase along the operably linked gene or mRNA of interest. Next 21-nucleotide phases of tasiRNA are generated by Dicer starting from a cleavage site of miRNA and target mRNA duplex. In some embodiments, the miRNA direct biogenesis of tasiRNA via 3′ initiation, see for example, Arabidopsis miR390.

In some embodiments, the miRNA target sequence operably linked to a target gene, mRNA, or cDNA sequence is introduced into a cell that expresses one or more miRNA that can bind to the miRNA target sequence and initiate tasiRNA biogenesis. The miRNA can be endogenous to the cell. The miRNA can also be an exogenous miRNA. In some embodiment the miRNA is incorporated into the genome of the cell. In some embodiments the miRNA is expressed from an extrachromosomal vector which is transfected into the cell. In some embodiments, the miRNA is expressed in vitro and transiently transfected into the cell, see for example, U.S. Published Application No. 2011/0165133. The miRNA sequence can also be operably linked to the polynucleotide encoding the miRNA target sequence and target gene sequence.

The sequence of the miRNA can be naturally occurring or a non-naturally occurring sequence. In some embodiments, the miRNA sequence is a sequence that occurs naturally in a plant. For example, the miRNA can be an miRNA sequence endogenous to soybean. Examples of isolated putative miRNA disclosed herein include: TCACCATTAAGACAGAGACCTT (SEQ ID NO:7), TAGGACTATGGTTTGGACAAGT (SEQ ID NO:8), TTGTTTTACCTATTCCACCCAT (SEQ ID NO:9), TTAATCAAGGAAATCACGGTCG (SEQ ID NO:10), TTAATCAAGGAAATCACGGTTG (SEQ ID NO:11), TTCATTTTTAAAATAGGCATTG (SEQ ID NO:12), and TGTTGTTTTACCTATTCCACCT (SEQ ID NO:13). The miRNA sequences are written as DNA, but one of skill in the art could readily convert the DNA sequence to RNA sequence. Thus, the RNA sequences of the miRNA are included within the scope of this application.

A polynucleotide is therefore disclosed having the nucleic acid sequence SEQ ID NO: 7, 8, 9, 10, 11, 12, 13 and complements and combinations thereof. A polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 7, 8, 9, 10, 11, 12, 13 complements or combinations thereof. A polynucleotide that hybridizes under stringent conditions to a polynucleotide consisting of the nucleic acid sequence SEQ ID NO: 7, 8, 9, 10, 11, 12, 13, complements or combinations thereof are also disclosed.

A polynucleotide that is a fragment of SEQ ID NO: 7, 8, 9, 10, 11, 12, 13 or combinations thereof are also disclosed. Therefore, a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of SEQ ID NO: 7, 8, 9, 10, 11, 12, 13, complements or combinations thereof is disclosed. The fragment can be at least 1, 2, 3, 4, 5, or more nucleotides shorter than SEQ ID NO: SEQ ID NO: 7, 8, 9, 10, 11, 12, 13.

Comparative genomic approaches and computational procedure to identify or predict miRNAs are described (for example the program MiRscan; Jones-Rhoades M W, Bartel D P (2004) Mol Cell 14(6):787-99, Lim et al. (2003) Genes Dev. 7(8):991-1008; Ohler U et al. (2004) RNA 10(9):1309-22; Robins H et al. (2005) Proc Natl Acad Sci USA 102(11):4006-9. Epub 2005 Feb. 28; Rhoades M W et al. (2002) Cell 110(4):513-20; Sunkar R et al. (2005) Plant Cell 17(5):1397-411. Epub 2005 Apr. 1; Wang X J et al. (2004) Genome Biol. 5(9):R65. Epub 2004 Aug. 31). Such prediction can also be made by EST analyses (Smalheiser N R (2003) Genome Biol. 4(7):403. Epub 2003 Jun. 18).

Other miRNA sequences suitable for use in the disclosed compositions and methods are known in the art, see for example, miRBase, release 18.

C. Constructs and Vectors

Vectors and constructs containing one or more miRNA target sequences operably linked to a polynucleotide of interest operably linked to an endogenous or heterologous expression control sequence are also disclosed. The constructs can include an expression cassette including, for example, one or more of the miRNA targets sequences of SEQ ID NO: 1, 2, 3, 4, 5, or 6.

Vectors and constructs containing one or more miRNA sequences operably linked to an endogenous or heterologous expression control sequence are also disclosed. The constructs can include an expression cassette including, for example, one or more of the miRNA sequences of SEQ ID NO: 7, 8, 9, 10, 11, 12, and 13.

In some embodiments a polynucleotide including the miRNA target sequence and the miRNA are expressed from the same vector.

The levels of expression for the miRNA, the construct containing the miRNA target sequence, or a combination thereof can be used for control of the gene-silencing process. For example, expression can be controlled by the promoter to high resulting in a high level of silencing, low resulting in a low level, or partial knockdown, etc. In some embodiments, expression of the miRNA, the construct containing the miRNA target sequence, or a combination thereof is inducible, tissue specific, or a combination thereof.

The production of small RNAs in a phased pattern means the sRNAs produced from the transgenic vector can be predicted, and so, avoiding the silencing of closely related genes is possible. Alternatively, if one would like to create a larger variety of sRNAs, one or more additional miRNA recognition sequences can be added to the transgenic vector to create a second phase. Additionally, the linear nature of these vectors makes them amenable to high-throughput cloning schemes.

1. Transformation Constructs

Transformation constructs can be engineered such that transformation of the nuclear genome and expression of transgenes from the nuclear genome occurs. Alternatively, transformation constructs can be engineered such that transformation of the plastid genome and expression of the plastid genome occurs.

Generally, nucleic acid sequences containing a miRNA target sequence operably linked to polynucleotide encoding a target gene or mRNA of interest are first assembled in expression cassettes behind a suitable promoter expressible in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors. Representative plant transformation vectors are described in plant transformation vector options available (Gene Transfer to Plants (1995), Potrykus, I. and Spangenberg, G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular biology—a laboratory course manual (1995), Maliga, P., Klessig, D. F., Cashmore, A. R., Gruissem, W. and Varner, J. E. eds. Cold Spring Laboratory Press, New York).

An additional approach is to use a vector to specifically transform the plant plastid chromosome by homologous recombination (U.S. Pat. No. 5,545,818 to McBride, et al.), in which case it is possible to take advantage of the prokaryotic nature of the plastid genome and insert a number of transgenes as an operon.

The following is a description of various components of typical expression cassettes.

2. Promoters

Plant promoters can be selected to control the expression of the transgene in different plant tissues or organelles, for all of which methods are known to those skilled in the art (Gasser & Fraley, Science 244:1293-99 (1989)). In a preferred embodiment, promoters are selected from those of plant or prokaryotic origin that are known to yield high expression in plastids. In certain embodiments the promoters are inducible. Inducible plant promoters are known in the art.

The transgenes can be inserted into an existing transcription unit (such as, but not limited to, psbA) to generate an operon. However, other insertion sites can be used to add additional expression units as well, such as existing transcription units and existing operons (e.g., atpE, accD). Such methods are described in, for example, U.S. Pat. App. Pub. 2004/0137631, which is incorporated herein by reference in its entirety. For an overview of other insertion sites used for integration of transgenes into the tobacco plastome, see Staub (Staub, J. M., “Expression of Recombinant Proteins via the Plastid Genome,” in: Vinci V A, Parekh S R (eds.) Handbook of Industrial Cell Culture: Mammalian, and Plant Cells, pp. 259-278, Humana Press Inc., Totowa, N.J. (2002)).

In general, the promoter can be from any class I, II or III gene. For example, any of the following plastidial promoters and/or transcription regulation elements can be used for expression in plastids. Sequences can be derived from the same species as that used for transformation. Alternatively, sequences can be derived from other species to decrease homology and to prevent homologous recombination with endogenous sequences.

For instance, the following plastidial promoters can be used for expression in plastids.

PrbcL promoter (Allison L A, Simon L D, Maliga P, EMBO J. 15:2802-2809 (1996); Shiina T, Allison L, Maliga P, Plant Cell 10: 1713-1722 (1998));

PpsbA promoter (Agrawal G K, Kato H, Asayama M, Shirai M, Nucleic Acids Research 29:1835-1843 (2001));

Prrn 16 promoter (Svab, et al., Proc. Natl. Acad. Sci. USA 90:913-917 (1993); Allison L A, Simon L D, Maliga P, EMBO J. 15:2802-2809 (1996));

PaccD promoter (Hajdukiewicz P T J, Allison L A, Maliga P, EMBO J. 16:4041-4048 (1997); WO 97/06250);

PclpP promoter (Hajdukiewicz P T J, Allison L A, Maliga P, EMBO J. 16:4041-4048 (1997); WO 99/46394);

PatpB, PatpI, PpsbB promoters (Hajdukiewicz P T J, Allison L A, Maliga P, EMBO J. 16:4041-4048 (1997));

PrpoB promoter (Liere K, Maliga P, EMBO J. 18:249-257 (1999));

PatpB/E promoter (Kapoor S, Suzuki J Y, Sugiura M, Plant J. 11:327-337 (1997)).

In addition, prokaryotic promoters (such as those from, e.g., E. coli or Synechocystis) or synthetic promoters can also be used.

Promoters vary in their strength, i.e., ability to promote transcription. Depending upon the host cell system utilized, any one of a number of suitable promoters known in the art may be used. For example, for constitutive expression, the CaMV 35S promoter, the rice actin promoter, or the ubiquitin promoter may be used. For example, for regulatable expression, the chemically inducible PR-1 promoter from tobacco or Arabidopsis may be used (see, e.g., U.S. Pat. No. 5,689,044 to Ryals, et al.).

A suitable category of promoters is that which is wound inducible. Numerous promoters have been described which are expressed at wound sites. Preferred promoters of this kind include those described by Stanford et al. Mol. Gen. Genet. 215: 200-208 (1989), Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), and Warner et al. Plant J. 3: 191-201 (1993).

Suitable tissue specific expression patterns include green tissue specific, root specific, stem specific, and flower specific. Promoters suitable for expression in green tissue include many which regulate genes involved in photosynthesis, and many of these have been cloned from both monocotyledons and dicotyledons. A suitable promoter is the maize PEPC promoter from the phosphoenol carboxylase gene (Hudspeth & Grula, Plant Molec. Biol. 12: 579-589 (1989)). A suitable promoter for root specific expression is that described by de Framond FEBS 290: 103-106 (1991); EP 0 452 269 to de Framond and a root-specific promoter is that from the T-1 gene. A suitable stem specific promoter is that described in U.S. Pat. No. 5,625,136 and which drives expression of the maize trpA gene.

The promoter can be a relatively weak plant expressible promoter. Thus, the promoter can in some embodiments initiate and control transcription of the operably linked nucleic acids about 10 to about 100 times less efficient than an optimal CaMV35S promoter. Relatively weak plant expressible promoters include the promoters or promoter regions from the opine synthase genes of Agrobacterium spp. such as the promoter or promoter region of the nopaline synthase, the promoter or promoter region of the octopine synthase, the promoter or promoter region of the mannopine synthase, the promoter or promoter region of the agropine synthase and any plant expressible promoter with comparably activity in transcription initiation. Other relatively weak plant expressible promoters may be dehiscence zone selective promoters, or promoters expressed predominantly or selectively in dehiscence zone and/or valve margins of fruits, such as the promoters described in WO97/13865.

Cis-regulatory elements from the promoter of photoperiod-responsive genes, coordinated motifs integrating hormones and stresses to photoperiod responses, and the promoters of photo-responsive genes such as those described in Mongkolsiriwatana C, Katsetsart J. (Nat. Sci.) 43: 164-177 (2009), can also be used.

An exemplary promoter is the GmUbi promoter used in the Examples below. It is known to be highly and constitutively expressed in soybean (Hernandez-Garcia, Martinelli, et al., 2009).

3. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

At the extreme 3′ end of the transcript, a polyadenylation signal can be engineered. A polyadenylation signal refers to any sequence that can result in polyadenylation of the mRNA in the nucleus prior to export of the mRNA to the cytosol, such as the 3′ region of nopaline synthase (Bevan, M., et al., Nucleic Acids Res., 11, 369-385 (1983)).

Another exemplary terminator is the terminator of the ribulose-1,5-bisphosphate carboxylase (Rubisco) small subunit (rbcS) gene.

4. Sequences for Expression Enhancement or Regulation

Numerous sequences have been found to enhance gene expression from within the transcriptional unit and these sequences can be used in conjunction with the genes to increase their expression in transgenic plants. For example, various intron sequences such as introns of the maize Adh1 gene have been shown to enhance expression, particularly in monocotyledonous cells. In addition, a number of non-translated leader sequences derived from viruses are also known to enhance expression, and these are particularly effective in dicotyledonous cells.

5. Coding Sequence Optimization

The coding sequence of the selected gene may be genetically engineered by altering the coding sequence for optimal expression (also referred to herein as “codon optimized”) in the crop species of interest. Methods for modifying coding sequences to achieve optimal expression in a particular crop species are well known (see, e.g. Perlak et al., Proc. Natl. Acad. Sci. USA 88: 3324 (1991); and Koziel et al, Biotechnol. 11: 194 (1993)). Therefore, in some embodiments, the disclosed nucleic acids sequences, or fragments or variants thereof, are genetically engineered for optimal expression in the crop species of interest.

6. Selectable Markers

Genetic constructs may encode a selectable marker to enable selection of plastid transformation events. There are many methods that have been described for the selection of transformed plants [for review see Miki, et al., Journal of Biotechnology 107:193-232 (2004) and references incorporated within]. Selectable marker genes that have been used extensively in plants include the neomycin phosphotransferase gene nptII (U.S. Pat. No. 5,034,322, U.S. Pat. No. 5,530,196), hygromycin resistance gene (U.S. Pat. No. 5,668,298), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. No. 5,463,175; U.S. Pat. No. 7,045,684). Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson, et al, Nat Biotechnol 22:455-458 (2004)). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants. Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein. Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296) whose references are incorporated in entirety. Improved versions of many of the fluorescent proteins have been made for various applications. Use of the improved versions of these proteins or the use of combinations of these proteins for selection of transformants will be obvious to those skilled in the art. It is also practical to simply analyze progeny from transformation events for the presence of the PHB thereby avoiding the use of any selectable marker.

For plastid transformation constructs, a preferred selectable marker is the spectinomycin-resistant allele of the plastid 16S ribosomal RNA gene (Staub J M, Maliga P, Plant Cell 4: 39-45 (1992); Svab Z, Hajdukiewicz P, Maliga P, Proc. Natl. Acad. Sci. USA 87: 8526-8530 (1990)). Selectable markers that have since been successfully used in plastid transformation include the bacterial aadA gene that encodes aminoglycoside 3′-adenyltransferase (AadA) conferring spectinomycin and streptomycin resistance (Svab, et al., Proc. Natl. Acad. Sci. USA 90:913-917 (1993)), nptII that encodes aminoglycoside phosphotransferase for selection on kanamycin (Carrer H, Hockenberry T N, Svab Z, Maliga P., Mol. Gen. Genet. 241: 49-56 (1993); Lutz K A, et al., Plant J. 37: 906-913 (2004); Lutz K A, et al., Plant Physiol. 145: 1201-1210 (2007)), aphA6, another aminoglycoside phosphotransferase (Huang F-C, et al, Mol. Genet. Genomics 268: 19-27 (2002)), and chloramphenicol acetyltransferase (Li, W., et al. (2010), Plant Mol Biol, DOI 10.1007/s11103-010-9678-4). Another selection scheme has been reported that uses a chimeric betaine aldehyde dehydrogenase gene (BADH) capable of converting toxic betaine aldehyde to nontoxic glycine betaine (Daniell H, et al., Curr. Genet. 39: 109-116 (2001)).

7. Targeting Sequences

The disclosed vectors and constructs may further include, within the region that encodes the protein to be expressed, one or more nucleotide sequences encoding a targeting sequence. A “targeting” sequence is a nucleotide sequence that encodes an amino acid sequence or motif that directs the encoded protein to a particular cellular compartment, resulting in localization or compartmentalization of the protein. Presence of a targeting amino acid sequence in a protein typically results in translocation of all or part of the targeted protein across an organelle membrane and into the organelle interior. Alternatively, the targeting peptide may direct the targeted protein to remain embedded in the organelle membrane. The “targeting” sequence or region of a targeted protein may contain a string of contiguous amino acids or a group of noncontiguous amino acids. The targeting sequence can be selected to direct the targeted protein to a plant organelle such as a nucleus, a microbody (e.g., a peroxisome, or a specialized version thereof, such as a glyoxysome) an endoplasmic reticulum, an endosome, a vacuole, a plasma membrane, a cell wall, a mitochondria, a chloroplast or a plastid. A chloroplast targeting sequence is any peptide sequence that can target a protein to the chloroplasts or plastids, such as the transit peptide of the small subunit of the alfalfa ribulose-biphosphate carboxylase (Khoudi, et al., Gene, 197:343-351 (1997)). A peroxisomal targeting sequence refers to any peptide sequence, either N-terminal, internal, or C-terminal, that can target a protein to the peroxisomes, such as the plant C-terminal targeting tripeptide SKL (Banjoko, A. & Trelease, R. N. Plant Physiol., 107:1201-1208 (1995); T. P. Wallace et al., “Plant Organellular Targeting Sequences,” in Plant Molecular Biology, Ed. R. Croy, BIOS Scientific Publishers Limited (1993) pp. 287-288, and peroxisomal targeting in plant is shown in M. Volokita, The Plant J., 361-366 (1991)).

Plastid targeting sequences are known in the art and include the chloroplast small subunit of ribulose-1,5-bisphosphate carboxylase Rubisco) (de Castro Silva Filho et al. Plant Mol. Biol. 30:769-780 (1996); Schnell et al. J. Biol. Chem. 266(5):3335-3342 (1991)); 5-(enolpyruvyl)shikimate-3-phosphate synthase (EPSPS) (Archer et al. J. Bioenerg. Biomemb. 22(6):789-810 (1990)); tryptophan synthase (Zhao et al. J. Biol. Chem. 270(11):6081-6087 (1995)); plastocyanin (Lawrence et al. J. Biol. Chem. 272(33):20357-20363 (1997)); chorismate synthase (Schmidt et al. J. Biol. Chem. 268(36):27447-27457 (1993)); and the light harvesting chlorophyll a/b binding protein (LHBP) (Lamppa et al. J. Biol. Chem. 263:14996-14999 (1988)). See also Von Heijne et al. Plant Mol. Biol. Rep. 9:104-126 (1991); Clark et al. J. Biol. Chem. 264:17544-17550 (1989); Della-Cioppa et al. Plant Physiol. 84:965-968 (1987); Romer et al. Biochem. Biophys. Res. Commun. 196:1414-1421 (1993); and Shah et al. Science 233:478-481 (1986). Alternative plastid targeting signals have also been described in the following: US 2008/0263728; Miras, S. et al. (2002), J Biol Chem 277(49): 47770-8; Miras, S. et al. (2007), J Biol Chem 282: 29482-29492.

8. Plants and Tissues for Transfection

Both dicotyledons (“dicots”) and monocotyledons (“monocots”) can be used in the disclosed positive selection system. Monocot seedlings typically have one cotyledon (seed-leaf), in contrast to the two cotyledons typical of dicots. Eudicots are dicots whose pollen has three apertures (i.e. triaperturate pollen), through one of which the pollen tube emerges during pollination. Eudicots contrast with the so-called ‘primitive’ dicots, such as the magnolia family, which have uniaperturate pollen (i.e. with a single aperture).

Monocots include one of the large divisions of Angiosperm plants (flowering plants with seeds protected within a vessel). They are herbaceous plants with parallel veined leaves and have an embryo with a single cotyledon, as opposed to dicot plants (dicotyledonous), which have an embryo with two cotyledons. Most of the important staple crops of the world, the so-called cereals, such as wheat, barley, rice, maize, sorghum, oats, rye and millet, are monocots. Thus, the plant can be a grass, such as wheat, barley, rice, maize, sorghum, oats, rye and millet.

The plant can therefore be a cereal crop such as wheat, oat, barley, or rice; a forage such as bahiagrass, dallisgrass, kleingrass, guineagrass, reed canarygrass, orchardgrass, ricegrass, foxtail, or vetch; a legume such as soybean, lentil, or chickpea; an oilseed such as canola; a vegetable such as onion or carrot; or a specialty crop such as caraway, hemp, or sesame.

In some embodiments, the plant is a soybean. For example, the soybean plant can be Jack, Resnik, Williams 82, Corsoy, Crawford, Hutcheson, Kunitz or Champ. Additional suitable soybean varieties are available from both academic and commercial institutions, such as—for example—the University of Guelph (Ontario Agricultural College; e.g. soybean varieties RCAT Staples, Westag 97, RCAT Bobcat, OAC Prudence, OAC Woodstock, OAC 9908), or soybean varieties from Daryland or Soygenetics. Additional suitable varieties are P1548402 (Peking), P1437654 (Er-hejjan), P1438489 (Chiquita), P1507354 (Tokei 421), P1548655 (Forrest), P1548988 (Pickett), P188788, P1404198 (Sun Huan Do), P1404166 (Krasnoaarmejkaja), Hartwig, Manokin, Doles, Dyer, and Custer.

In some embodiments, the plant is a miscanthus. Thus, the plant can be of the species Miscanthus floridulus, Miscanthus×giganteus, Miscanthus sacchariflorus (Amur silver-grass), Miscanthus sinensis, Miscanthus tinctorius, or Miscanthus transmorrisonensis.

Additional representative plants useful in the compositions and methods disclosed herein include the Brassica family including sp. napus, rapa, oleracea, nigra, carinata and juncea; industrial oilseeds such as Camelina sativa, Crambe, Jatropha, castor; Arabidopsis thaliana; soybean; cottonseed; sunflower; palm; coconut; rice; safflower; peanut; mustards including Sinapis alba; sugarcane and flax.

Crops harvested as biomass, such as silage corn, alfalfa, switchgrass, or tobacco, also are useful with the methods disclosed herein. Representative tissues for transformation using these vectors include protoplasts, cells, callus tissue, leaf discs, pollen, and meristems.

### II. Methods of Use

A. Methods of Gene Silencing in Plants

The disclosed compositions are typically used to reduce, inhibit, or silence gene expression in a plant, or part thereof. The methods of reducing, inhibiting or silencing gene expression disclosed herein typically, include introducing a polynucleotide including an miRNA target sequence operably linked to a sequence encoding a polynucleotide of interest into a cell. The miRNA target sequence operably linked to a sequence encoding a polynucleotide of interest can be transcribed in vitro and transiently transfected into the cell. Such methods are known in the art, see for example U.S. Published Application No. 2011/0165133. In some embodiments the miRNA target sequence operably linked to a sequence encoding a polynucleotide of interest are expressed from an expression construct. In preferred embodiments, the miRNA target sequence operably linked to a sequence encoding a polynucleotide of interest inserted into a plant vector, which can be transformed into the plant cell. In some embodiments, the miRNA target sequence operably linked to a sequence encoding a polynucleotide of interest is integrated into the nuclear or an organelle genome of the plant. In the some embodiments the construct is expressed extra-chromosomally.

To induce gene silencing in a plant, the miRNA target sequence operably linked to a sequence encoding a polynucleotide of interest is typically co-expressed with an miRNA specific for the target miRNA sequence as discussed in detail above. In some embodiments, the cell expresses the miRNA. The miRNA can be endogenous miRNA that is expressed, or can be induced to be expressed by the plant cell. Therefore, in some embodiments the heterologous construct is not needed to co-express the miRNA and induce tsaiRNA.

The miRNA can also be a heterologous miRNA. In some embodiments, the miRNA is expressed from a heterologous construct. Similar to the construct containing the miRNA target sequence operably linked to a sequence encoding a polynucleotide of interest, the miRNA can be transiently transfected into the cell, or expressed from a vector. The miRNA can integrated into the nuclear or an organelle genome of the plant, or expressed extra-chromosomally.

As discussed above, when co-expressed, the miRNA will bind to the miRNA target sequence and induce generation of tasiRNA which mediate gene silencing of the target gene of interest.

It is believed that a polynucleotide including an miRNA target sequence operably linked to a sequence encoding a polynucleotide of interest produced by transcription in a host organism such as a plant, can spread systemically throughout the organism. Therefore it is possible to reduce the phenotypic expression of a nucleic acid in cells of a non-transgenic scion of a plant grafted onto a transgenic stock including the polynucleotide or vice versa) a method which may be important in horticulture, viticulture or in fruit production.

As discussed in more detail below, the compositions and methods disclosed herein can be used to create transgenic plants inducible or stably silenced genes of interest. The genes of interest are generally related to one or more functions or pathways. Functions and pathways that can be modulated according to the disclosed compositions and methods include those disclosed in U.S. Published Application No. 2010/0192237.

1. Resistance to Plant Pathogens

In some embodiments, the methods are useful for inducing pathogen (e.g., virus or nematode) resistance, in eukaryotic cells or organisms, particularly in plant cells and plants. A resistance to plant pathogens such as arachnids, fungi, insects, nematodes, protozoans, viruses, bacteria and diseases can be achieved by reducing the gene expression of genes which are essential for the growth, survival, certain developmental stages (for example pupation) or the multiplication of a certain pathogen. A suitable reduction can bring about a complete inhibition of the above steps, but also a delay of one or more steps. This may be plant genes which, for example, allow the pathogen to enter, but may also be pathogen-homologous genes. Preferably, the tasiRNA produced according to the disclosed compositions and methods is directed against genes of the pathogen. For example, plants can be treated with suitable formulations of abovementioned agents, for example sprayed or dusted, the plants themselves, however, may also comprise the agents in the form of a transgenic organism and pass them on to the pathogens, for example in the form of a stomach poison. Various essential genes of a variety of pathogens are known to the skilled worker (for example for nematode resistance: WO 93/10251, WO 94/17194). Target gene sequences useful for inducing nematode resistance can also be found in U.S. Pat. No. 7,576,261, and U.S. Published Application No. 2009/0012029 which are incorporated by reference in their entirety.

Therefore, in some embodiments, the small RNAs produced according to the disclosed compositions and methods targets a gene that encodes a protein in a plant pathogen (e.g., an insect or nematode). The method can include introducing into the genome of a pathogen-targeted plant a nucleic acid construct that induces tasiRNA in an effective amount for reducing expression of a target gene within the pathogen when the pathogen (e.g., insect or nematode) ingests or infects cells from said plant. In a preferred embodiment, the gene suppression is fatal to the pathogen. Exemplary pathogens include fungal or bacterial pathogens such as Phytophthora infestans, Fusarium nivale, Fusarium graminearum, Fusarium culmorum, Fusarium oxysporum, Blumeria graminis, Magnaporthe grisea, Sclerotinia sclerotium, Septoria nodorum, Septoria tritici, Alternaria brassicae, Phoma lingam, bacterial pathogens such as Corynebacterium sepedonicum, Erwinia carotovora, Erwinia amylovora, Streptomyces scabies, Pseudomonas syringae pv. tabaci, Pseudomonas syringae pv. phaseolicola, Pseudomonas syringae pv. tomato, Xanthomonas campestris pv. malvacearum and Xanthomonas campestris pv. oryzae, and nematodes such as Globodera rostochiensis, G. pallida, Heterodera schachtii, Heterodera avenae, Ditylenchus dipsaci, Anguina tritici, Meloidogyne hapla, Heterodera glycines, Meloidogyne hapla, Meloidogyne arenaria, Meloidogyne incognita, and Ptratylenchus spp.

Resistance to viruses can be obtained for example by reducing the expression of a viral coat protein, a viral replicase, a viral protease and the like. A large number of plant viruses and suitable target genes are known in the art. Methods for obtaining pathogen resistant organisms, particularly plants, are also provided.

2. Abiotic Stress Factors

In some embodiments, the disclosed compositions and methods are used to protect an organism, such as a plant, against abiotic stress factors, including but not limited to, heat, chill, drought, increased moisture, environmental toxins, and UV radiation.

3. Biosynthetic Pathways

In some embodiments, the disclosed compositions and methods are used to modify a biosynthetic pathway in a plant.

a. Modification of the Composition or Content of Fatty Acids, Lipids or Oils

A modification of the fatty acid contents or the fatty acid composition, preferably in an oil crop such as oilseed rape or sunflower, can be achieved, for example, by reducing the gene expression of fatty acid biosynthesis genes, preferably those selected from the group consisting of genes encoding acetyl transacylases, acyl transport proteins (“acyl carrier protein”), desaturases such as stearyl desaturases or microsomal D12-desaturases, in particular Fad2-1 genes, malonyl transacylase, -ketoacyl-ACP synthetases, 3-keto-ACP reductases, enoyl-ACP hydrases, thioesterases such as acyl-ACP thioesterases, enoyl-ACP reductases. Various further advantageous approaches for modifying the lipid composition are described (Shure M et al. (1983) Cell 35:225-233; Preiss et al. (1987) Tailoring Genes for Crop Improvement (Bruening et al., eds.), Plenum Press, S.133-152; Gupta et al. (1988) Plant Mol. Biol. 10:215-224; Olive et al. (1989) Plant Mol Biol 12:525-538; Bhattacharyya et al. (1990) Cell 60:155-122; Dunwell J M (2000) J Exp Botany 51Spec No:487-96; Brar D S et al. (1996) Biotech Genet. Eng Rev 13:167-79; Kishore G M and Somerville C R (1993) Curr Opin Biotech 4(2):152-8). Preferred are, in particular, Fad2 genes (for example those described by Genbank Acc. No.: AF124360 (Brassica carinata), AF042841 (Brassica rapa), L26296 (Arabidopsis thaliana), A65102 (Corylus avellana)). Further advantageous genes and methods for modifying the lipid content are described, for example, in U.S. Pat. No. 5,530,192 and WO 94/18337. An elevated lipid content can also be achieved by reducing the starch content, for example as the result of the reduced expression of enzymes of the carbohydrate metabolism (for example ADP-glucose pyrophosphorylases).

b. Modification of the Carbohydrate Composition

A modification of the carbohydrate composition can be achieved for example by reducing the gene expression of carbohydrate metabolism genes or of carbohydrate biosynthesis genes, for example genes of the biosynthesis of amylose, pectins, cellulose or cell-wall carbohydrates. A multiplicity of cellular processes (maturation, storability, starch composition or starch content and the like) can thereby be influenced in an advantageous manner. Target genes which may be mentioned by way of example, but not by limitation, are phosphorylases, starch synthetases, Q-enzymes, sucrose-6-phosphate synthetases, sucrose-6-phosphate phosphatases, ADP-glucose pyrophosphorylases, branching enzymes, debranching enzymes and various amylases. The corresponding genes are described (Dunwell J M (2000) J Exp Botany 51Spec No:487-96; Brar D S et al. (1996) Biotech Genet Eng Rev 13:167-79; Kishore G M and Somerville C R (1993) Curr Opin Biotech 4(2):152-8). Advantageous genes for influencing the carbohydrate metabolism—in particular starch biosynthesis—are described in WO 92/11375, WO 92/11376, U.S. Pat. No. 5,365,016 and WO 95/07355.

c. Other Biosynthetic Pathways

Reduction of the susceptibility to bruising of, for example, potatoes by reducing for example polyphenol oxidase (WO 94/03607) and the like. Enhancement of vitamin E biosynthesis can be accomplished, for example by reducing the expression of genes from the homogentisate catabolic pathway such as, for example, homogentisate 1,2-dioxygenase (HGD; EC No.: 1.13.11.5), maleyl-acetoacetate isomerase (MAAI; EC No.: 5.2.1.2.) or fumaryl-acetoacetate hydrolase (FAAH; EC No.: 3.7.1.2). 3.1.2.17. Reduction of the nicotine content for example in tobacco can be accomplished by reducing expression of, for example, N-methyl-putrescin oxidase and putrescin N-methyltransferase. Reduction of the caffeine content in coffee bean (e.g., Coffea arabica) can be accomplished by reducing the gene expression of genes of caffeine biosynthesis such as 7-methylxanthine 3-methyltransferase. Reduction of the theophylline content in tea (Camellia sinensis) can be accomplished by reducing the gene expression of genes of theophylline biosynthesis such as, 1-methylxanthine 3-methyltransferase. Increasing of the methionine content by reducing threonine biosynthesis, can be accomplished by reducing the expression of threonine synthase (Zeh M et al. (2001) Plant Physiol 127(3):792-802).

The methods and compounds disclosed herein can be used for obtaining shatter resistance (WO 97/13865), for obtaining modified flower color patterns (EP 522 880, U.S. Pat. No. 5,231,020), for reducing the presence of unwanted (secondary) metabolites in organisms, such as glucosinolates (WO97/16559) or chlorophyll content (EP 779 364) in plants, for modifying the profile of metabolites synthesized in a eukaryotic cell or organisms by metabolic engineering e.g. by reducing the expression of particular genes involved in carbohydrate metabolism (WO 92/11375, WO 92/11376, U.S. Pat. No. 5,365,016, WO 95/07355) or lipid biosynthesis (WO 94/18337, U.S. Pat. No. 5,530,192) etc. Further examples of advantageous genes are mentioned for example in Dunwell, J Exp Bot. 2000; 51 Spec No; pages 487-96.

4. Modification of the Color or Pigmentation

A modification of the color or pigmentation, preferably of ornamentals, can be achieved for example by reducing the gene expression of flavonoid biosynthesis genes such as, for example, the genes of chalcone synthases, chalcone isomerases, phenylalanine ammonia lyases, dehydrokaempferol(flavone) hydroxylases such as flavanone 3-hydroxylases or flavanone 2-hydroxylases, dihydroflavonol reductases, dihydroflavanol 2-hydroxylases, flavonoid 3′-hydroxylases, flavonoid 5′-hydroxylases, flavonoid glycosyltransferases (for example glucosyltransferases such as UDPG:flavonoid 3-O-glucosyltransferases, UDPG: flavonol 7-O-glucosyltransferases or rhamnosyltransferases), flavonoid methyltransferases (such as, for example, SAM:anthocyanidin-3-(p-coumaroyl)rutinoside-5-glucoside-3′,5′-O-methyltransferases) and flavonoid acyltransferases (Hahlbrock (1981) Biochemistry of Plants, Vol. 7, Conn (Ed.); Weiring and de Vlaming (1984) “Petunia”, KC Sink (Ed.), Springer-Verlag, New York). Particularly suitable are the sequences described in EP-A1522 880.

5. Reduction of the Storage Protein Content

The reduction of the gene expression of genes encoding storage proteins (SP hereinbelow) has a large number of advantages such as, for example, the reduction of the allergenic potential or modification in the composition or quantity of other metabolites. Storage proteins are described, inter alia, in EP-A 0 591 530, WO 87/47731, WO 98/26064, EP-A 0 620 281; Kohno-Murase J et al. (1994) Plant Mol Biol 26(4): 1115-1124. SP serves for the storage of carbon, nitrogen and sulfur, which are required for the rapid heterotrophic growth in the germination of seeds or pollen. In most cases, they have no enzymatic activity. SP are synthesized in the embryo only during seed development and, in this process, accumulate firstly in protein storage vacuoles (PSV) of differently differentiated cells in the embryo or endosperm. Storage proteins can be classified into subgroups, as the function of further characteristic properties, such as, for example, their sedimentation coefficient or the solubility in different solutions (water, saline, alcohol). The sedimentation coefficient can be determined by means of ultracentrifugation in the manner with which the skilled worker is familiar (for example as described in Correia J J (2000) Methods in Enzymology 321:81-100). In total, four large gene families for storage proteins can be assigned, owing to their sequences: 2S albumins (napin-like), 7S globulins (phaseolin-like), 11S/12S globulins (legumin/cruciferin-like) and the zein prolamins 2S albumins are found widely in seeds of dicots, including important commercial plant families such as Fabaceae (for example soybean), Brassicaceae (for example oilseed rape), Euphorbiaceae (for example castor-oil plant) or Asteraceae (for example sunflower). 2S albumins are compact globular proteins with conserved cysteine residues which frequently form heterodimers. 7S globulins occur in trimeric form and comprise no cysteine residues. After their synthesis, they are cleaved into smaller fragments and glycosylated, as is the case with the 2S albumins.

Despite differences in polypeptide size, the different 7S globulins are highly conserved and can probably be traced to a shared precursor protein, as is the case with the 2S albumins. Only small amounts of the 7S globulins are found in monocots. In dicots, they always amount to less than the 11S/12S globulins. 11S/12S globulins constitute the main fraction of the storage proteins in dicots, in addition to the 2S albumins. The high degree of similarity of the different 11 S globulins from different plant genera, in turn, allow the conclusion of a shared precursor protein in the course of evolution. The storage protein is preferably selected from the classes of the 2S albumins (napin-like), 7S globulins (phaseolin-like), 11S/12S globulins (legumin/cruciferin-like) or zein prolamins. Especially preferred 11S/12S globulins comprise preferably 11S globulins from oilseed rape, soybean and Arabidopsis, sunflower, linseed, sesame, safflower, olive tree, soybean or various nut species. Especially preferred zein prolamins preferably comprise those from monocotyledonous plants, in particular maize, rice, oats, barley or wheat.

6. Prevention of Stem Break

In some embodiments, the disclosed compositions and methods are used to reduce or prevent stem break in a plant. A reduced susceptibility to stem break can be obtained for example by reducing the gene expression of genes of the carbohydrate metabolism (see above). Advantageous genes are described (WO 97/13865, inter alia) and comprise tissue-specific polygalacturonases or cellulases.

7. Delay of Fruit Maturation

In some embodiments, the disclosed compositions and methods are used to delay fruit maturation in a plant. Delayed fruit maturation can be achieved for example by reducing the gene expression of genes selected from the group consisting of polygalacturonases, pectin esterases, -(1-4)glucanases (cellulases), -galactanases-galactosidases), or genes of ethylene biosynthesis, such as 1-aminocyclopropane-1-carboxylate synthase, genes of carotenoid biosynthesis such as, for example, genes of prephytoene or phytoene biosynthesis, for example phytoene desaturases. Further advantageous genes are, for example, in WO 91/16440, WO 91/05865, WO 91/16426, WO 92/17596, WO 93/07275 or WO 92/04456, U.S. Pat. No. 5,545,366).

8. Inducing Male Sterility

In some embodiments, the disclosed compositions and methods are used to induce male sterility in a plant. Suitable target genes are described in WO 94/29465, WO89/10396, and WO 92/18625. A particular application for reduction of the phenotypic expression of a transgene in a plant cell, has been described for the restoration of male fertility, the latter being obtained by introduction of a transgene comprising a male sterility DNA (WO 94/09143, WO 91/02069). The nucleic acid of interest is specifically the male sterility DNA.

9. Reduction of Undesired or Toxic Plant Constituents

In some embodiments, the disclosed compositions and methods are used to reduction of undesired or toxic plant constituents in a plant. Reduction of undesired or toxic plant constituents such as, for example, glucosinolates. Suitable target genes are described (in WO 97/16559).

10. Delay of Senescence Symptoms

In some embodiments, the disclosed compositions and methods are used to delay of senescence symptoms in a plant. Suitable target genes include, but are not limited to, cinnamoyl-CoA:NADPH reductases or cinnamoyl alcohol dehydrogenases. Further target genes are described (in WO 95/07993).

11. Modification of the Lignification or the Lignin

In some embodiments, the disclosed compositions and methods are used to modify the lignification or the lignin content in a plant. Suitable target genes are described in WO 93/05159, WO 93/05160.

12. Modification of Plant Structure

In some embodiments, the disclosed compositions and methods are used to modify plant structure, for example branching, lateral rooting, fiber content, or quality in a plant. Target genes and methods of altering branching and root architecture are described in U.S. Published Application No. 2011/0191910, U.S. Published Application No. 2005/0177901, and U.S. Published Application No. 2011/0231959. Modification of the fiber content in foodstuffs, preferably in seeds, can be accomplished by reducing the expression of coffeic acid O-methyltransferase or of cinnamoyl alcohol dehydrogenase. Modification of the fiber in plants such as cotton can be accomplished by targeting genes such as those described in U.S. Pat. No. 5,597,718.

B. Plant Transformation Techniques

The transformation of suitable agronomic plant hosts using vectors expressing transgenes can be accomplished with a variety of methods and plant tissues. Representative transformation procedures include Agrobacterium-mediated transformation, biolistics, microinjection, electroporation, polyethylene glycol-mediated protoplast transformation, liposome-mediated transformation, and silicon fiber-mediated transformation (U.S. Pat. No. 5,464,765 to Coffee, et al.; “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga et al. eds.) Cold Spring Laboratory Press, New York (1995)).

Plants can be transformed by a number of reported procedures (U.S. Pat. No. 5,015,580 to Christou, et al.; U.S. Pat. No. 5,015,944 to Bubash; U.S. Pat. No. 5,024,944 to Collins, et al.; U.S. Pat. No. 5,322,783 to Tomes et al.; U.S. Pat. No. 5,416,011 to Hinchee et al.; U.S. Pat. No. 5,169,770 to Chee et al.). A number of transformation procedures have been reported for the production of transgenic maize plants including pollen transformation (U.S. Pat. No. 5,629,183 to Saunders et al.), silicon fiber-mediated transformation (U.S. Pat. No. 5,464,765 to Coffee et al.), electroporation of protoplasts (U.S. Pat. No. 5,231,019 Paszkowski et al.; U.S. Pat. No. 5,472,869 to Krzyzek et al.; U.S. Pat. No. 5,384,253 to Krzyzek et al.), gene gun (U.S. Pat. No. 5,538,877 to Lundquist et al. and U.S. Pat. No. 5,538,880 to Lundquist et al.), and Agrobacterium-mediated transformation (EP 0 604 662 A1 and WO 94/00977 both to Hiei Yukou et al.). The Agrobacterium-mediated procedure is particularly preferred as single integration events of the transgene constructs are more readily obtained using this procedure which greatly facilitates subsequent plant breeding. Cotton can be transformed by particle bombardment (U.S. Pat. No. 5,004,863 to Umbeck and U.S. Pat. No. 5,159,135 to Umbeck). Sunflower can be transformed using a combination of particle bombardment and Agrobacterium infection (EP 0 486 233 A2 to Bidney, Dennis; U.S. Pat. No. 5,030,572 to Power et al.). Flax can be transformed by either particle bombardment or Agrobacterium-mediated transformation. Switchgrass can be transformed using either biolistic or Agrobacterium mediated methods (Richards et al. Plant Cell Rep. 20: 48-54 (2001); Somleva et al. Crop Science 42: 2080-2087 (2002)). Methods for sugarcane transformation have also been described (Franks & Birch Aust. J. Plant Physiol. 18, 471-480 (1991); WO 2002/037951 to Elliott, Adrian, Ross et al.).

Recombinase technologies which are useful in practicing the current invention include the cre-lox, FLP/FRT and Gin systems. Methods by which these technologies can be used for the purpose described herein are described for example in (U.S. Pat. No. 5,527,695 to Hodges et al.; Dale and Ow, Proc. Natl. Acad. Sci. USA, 88:10558-10562 (1991); Medberry et al., Nucleic Acids Res., 23: 485-490 (1995)).

Engineered minichromosomes can also be used to express one or more genes in plant cells. Cloned telomeric repeats introduced into cells may truncate the distal portion of a chromosome by the formation of a new telomere at the integration site. Using this method, a vector for gene transfer can be prepared by trimming off the arms of a natural plant chromosome and adding an insertion site for large inserts (Yu et al. 2007, Yu et al. 2006). The utility of engineered minichromosome platforms has been shown using Cre/lox and FRT/FLP site-specific recombination systems on a maize minichromosome where the ability to undergo recombination was demonstrated (Yu, Han, Gao, Vega and Birchler 2007, Yu, Lamb, Han and Birchler 2006). Such technologies could be applied to minichromosomes, for example, to add genes to an engineered plant. Site specific recombination systems have also been demonstrated to be valuable tools for marker gene removal (Kerbach, S. et al., Theor. Appl. Genet. 111:1608-1616 (2005)), gene targeting (Chawla, R. et al., Plant Biotechnol. J, 4:209-218 (2006); Choi, S. et al., Nucleic Acids Res., 28, E19 (2000); Srivastava V & Ow D W, Plant Mol Biol. 46:561-566 (2001); Lyznik L A et al., Nucleic Acids Res., 21: 969-975 (1993)) and gene conversion (Djukanovic V et al., Plant Biotechnol J., 4:345-357 (2006).

An alternative approach to chromosome engineering in plants involves in vivo assembly of autonomous plant minichromosomes (Carlson et al. 2007). Plant cells can be transformed with centromeric sequences and screened for plants that have assembled autonomous chromosomes de novo. Useful constructs combine a selectable marker gene with genomic DNA fragments containing centromeric satellite and retroelement sequences and/or other repeats.

Another approach useful to the described invention is Engineered Trait Loci (“ETL”) technology (U.S. Pat. No. 6,077,697; US Patent Application 2006/0143732). This system targets DNA to a heterochromatic region of plant chromosomes, such as the pericentric heterochromatin, in the short arm of acrocentric chromosomes. Targeting sequences may include ribosomal DNA (rDNA) or lambda phage DNA. The pericentric rDNA region supports stable insertion, low recombination, and high levels of gene expression. This technology is also useful for stacking of multiple traits in a plant (US Patent Application 2006/0246586).

Zinc-finger nucleases (ZFNs) are also useful for practicing the invention in that they allow double strand DNA cleavage at specific sites in plant chromosomes such that targeted gene insertion or deletion can be performed (Shukla, et al., Nature 459(7245):437-31 (2009); Townsend, et al., Nature 459(7245):442-5 (2009)).

Following transformation by any one of the methods described above, the following procedures can, for example, be used to obtain a transformed plant expressing the transgenes: select the plant cells that have been transformed on a selective medium, regenerate the plant cells that have been transformed to produce differentiated plants, select transformed plants expressing the transgene producing the desired level of desired polypeptide(s) in the desired tissue and cellular location.

Transformation techniques for dicotyledons are well known in the art and include Agrobacterium-based techniques and techniques that do not require Agrobacterium. Non-Agrobacterium techniques involve the uptake of heterologous genetic material directly by protoplasts or cells. This is accomplished by PEG or electroporation mediated uptake, particle bombardment-mediated delivery, or microinjection. In each case the transformed cells may be regenerated to whole plants using standard techniques known in the art.

Transformation of most monocotyledon species has now become somewhat routine. Preferred techniques include direct gene transfer into protoplasts using PEG or electroporation techniques, particle bombardment into callus tissue or organized structures, as well as Agrobacterium-mediated transformation.

Plants from transformation events are grown, propagated and bred to yield progeny with the desired trait, and seeds are obtained with the desired trait, using processes well known in the art.

C. Plastid Transformation

In another embodiment the transgene is directly transformed into the plastid genome. Plastid transformation technology is extensively described in U.S. Pat. No. 5,451,513 to Maliga et al., U.S. Pat. No. 5,545,817 to McBride et al., and U.S. Pat. No. 5,545,818 to McBride et al., in PCT application no. WO 95/16783 to McBride et al., and in McBride et al. Proc. Natl. Acad. Sci. USA 91, 7301-7305 (1994). The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Suitable plastids that can be transfected include, but are not limited to, chloroplasts, etioplasts, chromoplasts, leucoplasts, amyloplasts, proplastids, statoliths, elaioplasts, proteinoplasts and combinations thereof.

D. Methods for Reproducing Transgenic Plants

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

In plastid transformation procedures, further rounds of regeneration of plants from explants of a transformed plant or tissue can be performed to increase the number of transgenic plastids such that the transformed plant reaches a state of homoplasmy (all plastids contain uniform plastomes containing transgene insert).

The cells that have been transformed may be grown into plants in accordance with conventional techniques. See, for example, McCormick et al. Plant Cell Reports 5:81-84(1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

In some scenarios, it may be advantageous to insert a multi-gene pathway into the plant by crossing of lines containing portions of the pathway to produce hybrid plants in which the entire pathway has been reconstructed. This is especially the case when high levels of product in a seed compromises the ability of the seed to germinate or the resulting seedling to survive under normal soil growth conditions. Hybrid lines can be created by crossing a line containing one or more the transgene miRNA targeting sequence constructs disclosed herein with a line containing the miRNA. Use of lines that possess cytoplasmic male sterility (Esser, K. et al., 2006, Progress in Botany, Springer Berlin Heidelberg. 67, 31-52) with the appropriate maintainer and restorer lines allows these hybrid lines to be produced efficiently. Cytoplasmic male sterility systems are already available for some Brassicaceae species (Esser, K. et al., 2006, Progress in Botany, Springer Berlin Heidelberg. 67, 31-52).

### EXAMPLES

### Example 1

### Identification of Putative tasiRNA Loci

### Materials and Methods

Identification of Putative ta-siRNA Loci

Putative PHAS loci were identified by aligning small RNA sequences from a soybean hairy root library to the soybean genome (Glyma v1.0) with the commercial software Geneious version 5.4 (Drummond, et al., Geneious (2011)). Regions with high coverage of reads were evaluated by a number of criteria. First the sRNA reads had to be primarily 21-nt in length, consistent with DCL4 activity. Second, reads had to be from both positive and negative strands, which would indicate the presence of dsRNA. Finally a single species of sRNA (presumably a miRNA) had to align 5′ to the majority of the aligned sequences as this would indicate the possibility of a miRNA inducing the production of the siRNAs. In order to get putative miRNAs to align to the reference sequence, the miRNA alignments allowed for mismatches. PHAS loci were also identified by aligning all miRNA from fabaceae deposited in miRBase release 18.0. Regions with siRNAs adjacent to possible miRNA cut sites were evaluated following the criteria outlined above. Once putative miRNA recognition sequences were identified, they were compared to the soybean genome via BLAST to identify other potential PHAS loci.

Known and Putative miRNA Tissue Specific Expression

Small RNA reads from previously published datasets (Zabala, Campos, et al., 2012) were assembled to the 22-nt putative miRNAs using an in house Bowtie script.

### Results

Small-RNA analysis identified nine putative PHAS loci from the soybean hairy root dataset. The results are summarized in Table 1. The sequences for the miRNAs are provided as DNA sequence. One of skill in the art could readily convert the DNA sequences in miRNA sequences. There are six unique miRNA recognition sequences targeting the nine putative PHAS loci. Six of the loci are putative protein coding genes, while four of the loci are located in intergenic regions. Zhai, et al., Genes & Development 25:2540-2553. (2011) predicted some of the same loci, with the exceptions being Glyma18g03980.2, Glyma17g2260.1 and Gm04:43,624,071 . . . 43,624,750, however, the target sequences were not identified.

All of the miRNAs identified as possible triggers of the phasiRNAs were 22-nt in length, which is consistent with studies in arabidopsis (Chen, et al., PNAS, 107: 15269-15274 (2010), Cuperus, et al., Nature Structural & Molecular Biology, 17: 997-U111 (2010) and soybean (Zhai, et al., Genes & Development 25:2540-2553. (2011)). The two miRNAs miR1510 and miR1509a are known miRNAs in soybean. One miRNA discovered in peanut (Arachis hypogaea L.), miR3514 has not been identified in soybean, however it potentially targets three loci in soybean. Many of the miRNAs identified were size variants of previously identified miRNAs. In miRBase release 18, miR1514a, miR1509b, miR1510a, and miR5770 are all annotated as 21-nt miRNAs. However, the 22-nt versions identified herein could be derived from the stem-loop precursor. Such iso-mIRs, are denoted with the 0.2 notation. The previously identified miRNAs, or the ones here may have been incorrectly processed during the bioinformatics pipelines resulting in the wrong size class, or the miRNAs identified here could be the result of alternative processing by the cellular machinery and have biological significance. One locus, Gm04:43,624,071 . . . 43,624,750 is potentially targeted by two miRNAs, miR1509a and miR1509b.2. The putative miRNA 1510 identified here cannot be produced from the known miR1510a and miR1510b loci as the 3′ terminal T is an A in the annotated miRNA loci.

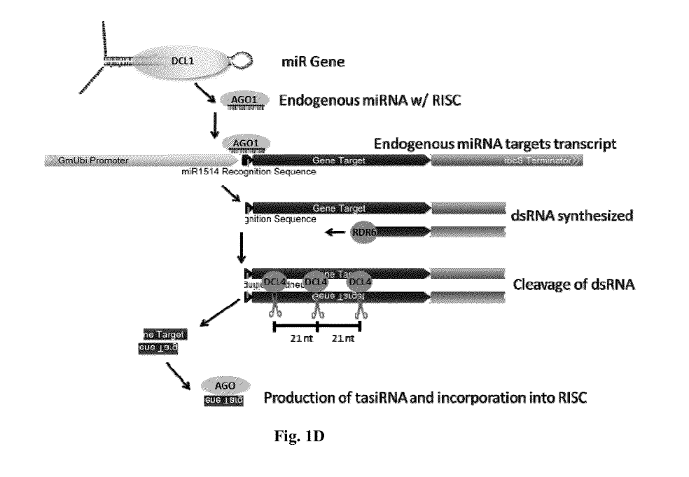

miRNAs are known to be differentially expressed. The expression patterns of the putative miRNAs identified in the analysis were examined Using soybean sRNA datasets from previously published studies and in this one, the number of sRNAs for each of the putative miRNAs 22-nt in length were quantified. A single library for each of the immature seed tissues and vegetative tissues and five hairy root libraries were analyzed. 1510a.2 and 1514a.2 are expressed at relatively low (0-20 counts per million reads) but consistent levels in most tissue types (FIG. 2). In contrast, 1509b.2 is highly expressed (250-400 counts per million reads) in all tissues except shoot tips, leaves, and hairy roots and peaks with as many as 900 counts per million reads in root tissue. 5770.2 is not present or is below 1 count per million reads in all tissues except hairy roots. This data indicates that the putative miRNAs identified here are differentially expressed in the tissue types evaluated.

The locus Gm04:45,906,669 . . . 45,909,231 was previously predicted to contain a target site for gma-miR2118 (Zhai, et al., Genes & Development 25:2540-2553. (2011)), however the data provided herein does not support this and instead indicates a miRNA species similar to gma-miR1510a-3p to be the phasiRNA-inducing miRNA.

Two loci that produce phased siRNA, Glyma16g01940.1 and Glyma07g05360.1, are homologs and in our dataset a putative miRNA species most similar to the miRNA gma-miR1514a appears to target the mRNA sequences. Gma-miR1514a (TTCATTTTTAAAATAGGCATT) (SEQ ID NO:14) is annotated as 21-nt in length (miRBase, release 18), however the species identified here is 22-nt in length and has an additional G at the 3′ end compared to gma-miR1514a (SEQ ID NO:14). This putative miRNA also has a mismatch compared to gma-miR1514b.

TABLE 1
Summary of tasiRNA Loci in Soybean Hairy Roots

| | | Putative | miRNA | PHAS | PHAS | |
|---|---|---|---|---|---|---|
| | | miRNA | recognition | Locus | Locus | |
| miRNA | Related | sequence | sequence | Glyma | Character- | PHAS Locus |
| Name | miRNA | 5′->3′ | 5′->3′ | model | ization | Coordinates |
| 3514 | Ahy | TCACCATT | AAGGTCTC | intergenic | uncharac- | Gm16: 30, |
| | 3514-3p | AAGACAGA | TGTCTTAA | | terized, | 415, 404.. |
| | | GACCTT | TGGTGA | | similar | 30, 421, |
| | | (SEQ ID | (SEQ ID | | to PPR | 646 |
| | | NO: 7) | NO: 1) | | protein | |
| 3514 | Ahy | TCACCATT | AAGGTCTC | Glyma18g03980.2 | 3′ UTR, | Gm18: 2, |
| | 3514-3p | AAGACAGA | TGTCTTAA | | protein of | 747, 299.. |
| | | GACCTT | TGGTGA | | unknown | 2, 748, |
| | | (SEQ ID | (SEQ ID | | function | 817 |
| | | NO: 7) | NO: 1) | | | |
| 3514 | Ahy | TCACCATT | AAGGTCTC | intergenic | MIR4409 | Gm16: 5, |
| | 3514-3p | AAGACAGA | TGTCTTAA | | and | 743, 468.. |
| | | GACCTT | TGGTGA | | MIR5372 | 5, 745, |
| | | (SEQ ID | (SEQ ID | | | 496 |
| | | NO: 7) | NO: 1) | | | |
| 5770.2 | Gm | TAGGACTA | TCTTGTCC | Glyma17g02260.1 | copper | Gm17: 1, |
| | 5770b | TGGTTTGG | AAACCATA | | amino | 453, 405.. |
| | | ACAAGT | GTCCAA | | oxidase | 1, 456, |
| | | (SEQ ID | (SEQ ID | | | 485 |
| | | NO: 8) | NO: 2) | | | |
| 1510a.2 | Gm | TTGTTTTA | ATGGGTGG | Glyma04g39740.1 | LRR | Gm04: 45, |
| | 1510a-3p | CCTATTCC | AATAGGGA | | protein | 906, 669.. |
| | | ACCCAT | AAACAA | | | 45, 909, |
| | | (SEQ ID | (SEQ ID | | | 231 |
| | | NO: 9) | NO: 3) | | | |
| 1509a | Gm | TTAATCAA | CAACCTTG | intergenic | no | |
| | 1509a | GGAAATCA | ATTTCCTT | | infor- | Gm04: 43, |
| | | CGGTTG | GATTAA | | mation | 624, 071.. |
| | | (SEQ ID | (SEQ ID | | | 43, 624, |
| | | NO: 10) | NO: 4) | | | 750 |
| 1509b.2 | Gm | TTAATCAA | CAACCTTG | intergenic | no | Gm04: 43, |
| | 1509b | GGAAATCA | ATTTCCTT | | infor- | 624, 071.. |
| | | CGGTTG | GATTAA | | mation | 43, 624, |
| | | (SEQ ID | (SEQ ID | | | 750 |
| | | NO: 11) | NO: 4) | | | |
| 1514a.2 | Gm | TTCATTTT | CAATGCCT | Glyma16g01940.1 | No Apical | Gm16: 1, |
| | 1514a | TAAAATAG | ATTTTAGA | | Meristem | 443, 765.. |
| | | GCATTG | AATGAA | | protein, | 1, 449, |
| | | (SEQ ID | (SEQ ID | | uncharac- | 622 |
| | | NO: 12) | NO: 5) | | terized | |
| 1514a.2 | Gm | TTCATTTT | CAATGCCT | Glyma07g05360.1 | No Apical | Gm07: 3, |
| | 1514a | TAAAATAG | ATTTTAGA | | Meristem | 999, 218.. |
| | | GCATTG | AATGAA | | protein, | 4, 001, |
| | | (SEQ ID | (SEQ ID | | uncharac- | 905 |
| | | NO: 12) | NO: 5) | | terized | |
| 1510 | Gm | TGTTGTTT | AGGTGGAA | Glyma12g27800.1 | LRR | Gm12: 31, |
| | 1510a-3p | TACCTATT | TAGGAAAA | | protein | 264, 569.. |
| | | CCACCT | ACAACT | | | 31, 268, |
| | | (SEQ ID | (SEQ ID | | | 587 |
| | | NO: 13) | NO: 6) | | | |

### Example 2

### Fusion of miR1514 Target Site Leads to Reduction of Target Transcript and Production of Secondary siRNAs Against Glyma02g43860 and Glyma07g14460

### Materials and Methods

Vector Design

A modified pPZP200 vector (Covert, et al., Mycological Research 105:259-264 (2001)) was used as the binary backbone, hereafter p201N. Four vectors (1514NFR, 1514P450, NFR, and P450) were made by amplifying the 356-bp NFR1a target region and the 343-bp P450 target region using Phusion® High Fidelity Polymerase (Finnzymes). The 5′ ends of the primers contain the restriction sites Asc I and Avr II for cloning. To make the 1514a.2 fusion vectors, a 1514a.2 target site was included on the forward primer. Using traditional cloning techniques, the amplicon was inserted into a modified pPZP200 vector (Covert, et al., Mycological Research 105:259-264 (2001)) with the GmUbi promoter (Chiera, et al., Plant Cell Reports 26:1501-1509 (2007)) and rbcs terminator (An, et al., Embo Journal 4:277-284 (1985)). Vectors were sequence-verified and electroporated into A. rhizogenes strain K599 cells (Savka, et al., Phytopathology 80:503-508 (1990)).

For the GFP silencing vectors, the target region was amplified as above, except each of the six putative miRNA recognition sequences were used in the same location as the 1514.

The Solanum tuberosum ubiquitin promoter StUbi drives the expression of the nptII selectable marker. The GmUbi promoter (Hernandez-Garcia, Martinelli, et al., 2009) drives the expression of the silencing and control cassettes and is terminated by the rbcs terminator (An, et al., Embo Journal 4:277-284 (1985)) (FIGS. 1B, 1C, and 1D). Gene-silencing targets were created by amplifying the target regions from cDNA or DNA with the miR1514a.2 recognition sequence (5′-CAATGCCTATTTTAGAAATGAA-3′ (SEQ ID NO:5)) fused to the forward primer (Table 2). Control vectors were created with the same primers, except with the miRNA recognition sequence omitted. The amplicons were inserted into the p201N vector via the AscI and AvrII restriction sites.

TABLE 2
Primer

| | | | PCR |
|---|---|---|---|
| | Forward | Reverse | efficiency |
| Primers for qRT | | | |
| NFR4 | GTCTTGATGCACAGTTGCTGCAGC | CTCGGGTACAATGGAACATAGTCTCC | 1.84 |
| | (SEQ ID NO: 15) | (SEQ ID NO: 16) | |
| GFP1 | GCCGACAAGCAGAAGAACGGCA | AGGTAGTGGTTGTCGGGCAGCA | 1.942 |
| | (SEQ ID NO: 17) | (SEQ ID NO: 18) | |
| P4501 | ACAGAAGCTGAAGTGACTGGGCTT | CACAGCTGAAAGGTACTGGTTGTT | 1.944 |
| | (SEQ ID NO: 19) | (SEQ ID NO: 20) | |
| Metalloprotease | Libault et al. | Libault et al. | 1.947 |
| rbcsT | GTTCGAGTATTATGGCATTGGGAAAACTG | CACAGTTCGATAGCGAAAACCGAAT | 1.962 |
| | (SEQ ID NO: 21) | (SEQ ID NO: 22) | |
| Cloning Primers | | | |
| 1514:GmNFR1a | ATATAGGCGCGCCCAATGCCTATTTTA | TATATCCTAGGCTCGCCTCTCAGTTCTG | |
| | GAAATGAACAGGTCTTGCTAGGGGTGC | CATAA | |
| | (SEQ ID NO: 23) | (SEQ ID NO: 24) | |
| 1514:GmP450 | ATATAGGCGCGCCCAATGCCTATTTTA | TATATCCTAGGCTGATTTGCTTGCACTT | |
| | GAAATGAACTTAGGGCCAACAAGCTCA | TTGCG | |
| | AGGG (SEQ ID NO: 25) | (SEQ ID NO: 26) | |
| GmNFR1a | ATATAGGCGCGCCCAGGTCTTGCTAGG | TATATCCTAGGCTCGCCTCTCAGTTCTG | |
| | GGTGC | CATAA | |
| | (SEQ ID NO: 27) | (SEQ ID NO: 28) | |
| GmP450 | ATATAGGCGCGCCCTTAGGGCCAACAA | TATATCCTAGGCTGATTTGCTTGCACTT | |
| | GCTCAAGGG | TTGCG | |
| | (SEQ ID NO: 29) | (SEQ ID NO: 30) | |
| 1509:GFP | ATATAGGCGCGCCCAACCTTGATTTCC | TATATCCTAGGGCCGTTCTTCTGCTTGT | |
| | TTGATTAACTCGTGACCACCTTCA | C | |
| | (SEQ ID NO: 31) | (SEQ ID NO: 32) | |
| 1510:GFP | ATATAGGCGCGCCAGGTGGAATAGGA | = | |
| | AAAACAACTCTCGTGACCACCTTCA | | |
| | (SEQ ID NO: 33) | | |
| 1510a.2:GFP | ATATAGGCGCGCCATGGGTGGAATAG | = | |
| | GGAAAACAACTCGTGACCACCTTCA | | |
| | (SEQ ID NO: 34) | | |
| 1514:GFP | ATATAGGCGCGCCCAATGCCTATTTTA | = | |
| | GAAATGAACTCGTGACCACCTTCA | | |
| | (SEQ ID NO: 35) | | |
| 3514:GFP | ATATAGGCGCGCCAAGGTCTCTGTCTT | = | |
| | AATGGTGACTCGTGACCACCTTCA | | |
| | (SEQ ID NO: 36) | | |
| 5770:GFP | ATATAGGCGCGCCTCTTGTCCAAACCA | = | |
| | TAGTCCAACTCGTGACCACCTTCA | | |
| | (SEQ ID NO: 37) | | |
| GFP | ATATAGGCGCGCCCTCGTGACCACCTT | = | |
| | CA | | |
| | (SEQ ID NO: 38) | | |
| 1514:GP | GGCCGCACCGGCGCGCCCAATGCCTAT | AACCTAGGAAGGATCCACTTTGGCCGC | |
| | TTTAGAAATGAACCATCAAGACGTTCTC | TTCGAGATCA | |
| | CGTGA (SEQ ID NO: 39) | (SEQ ID NO: 40) | |
| Genotyping Primers | | | |
| GmUbi842 F | CGAGATTGCTTCAGATCCGTA | | |
| | (SEQ ID NO: 41) | | |
| rbcsT272 R | CGGTCATTAGAGGCCACGAT | | |
| | (SEQ ID NO: 42) | | |

Restriction sites in bold.
miRNA recognition sequences underlined

Hairy Root Transformation of Soybean

Soybean cultivar ‘Jack’ seeds were transformed with A. rhizogenes strain K599 harboring the vectors of interest with slight modifications from the protocol previously described (Cho, et al., Planta 210:195-204 (2000)). Briefly, soybean seeds were germinated for approximately one week under sterile conditions on a filter paper wetted with ½ MS liquid medium (½ MS salts, B5 vitamins, 30 g L-1 sucrose). Agrobacterium from glycerol stocks were streaked out on YM medium (Lin J. J., Plant Science 101:11-15 (1994) supplemented with 50 mg L-1 kanamycin.

Soybean cotyledons were prepared in a manner similar to that for cot-node transformation (Olhoft, et al., Planta 216:723-735 (2003)); the root and lower hypocotyl is removed from the cotyledons, leaving approximately 5 mm of hypocotyl. The apical shoot and hypocotyl are cut longitudinally to produce two mirror image cotyledons with a short hypocotyl piece. The meristem is removed and the cotyledons are cut 1 mm deep on the adaxial surface with a scalpel dipped in a solution of Agrobacterium (solution is PB Buffer+100 μM acetosyringone). Cotyledons are co-cultivated with the Agrobacterium for 3 days on filter paper wetted with 2 mL of ½ MS+100 μM Acetosyringone. Cotyledons are then transferred to medium according to Cho, et al., Planta 210:195-204 (2000) with the following modifications: ½ MS salts, 2 g L-1 Phytagel, and 500 mg L-1 timentin to inhibit A. rhizogenes.

Each root is treated as an individual event and transferred to HRG medium with 10 mg L-1 of the selective agent, Geneticin (G418), which selects for the nptII gene. No-vector control roots are grown on HRG medium without G418. Those roots that grow on HRG+G418 medium are considered events, and a 2-cm portion of a root tip is collected for CTAB DNA extraction (Murray, et al., Nucleic Acids Research 8:4321-4325 (1980)). PCR is performed to confirm the insertion of the gene of interest using primers in the promoter and terminator (Table 2). After selection and PCR verification, roots are grown on individual plates for two weeks. Root tissue is then harvested and frozen in liquid nitrogen.

RNA Extraction and Quantitative RT-PCR

Total RNA was extracted from frozen roots with Tri-Reagent and treated with Turbo DNase™ (Ambion®) to remove all contaminating DNA. 100 ng of treated total RNA was used as the template in the Go Taq® 1-Step RT-qPCR system from Promega Corporation. The qRT-PCR reaction was performed in a Light Cycler 480II according to kit instructions. The metalloprotease amplicon, Libault, et al., The Plant Genome 1:44-54 (2008), was used to normalize expression of each of the target genes. A list of primers used and amplicon efficiencies can be found in Table 2 above.

Small RNA Sequencing and Assembly

Small RNA libraries were prepared from total RNA using Illumina's TruSeq™ small RNA library kit. The MiSeq was used for small RNA sequencing according to manufacturer instructions. Raw reads were separated by barcodes using the MiSeq Reporter software. Fastq files were uploaded to Geneious where adapters were trimmed and reads 18-25 nt in length were selected for assembly. The reads were assembled to the respective silencing or control cassettes and the target mRNA using the following conditions: Gaps not allowed, word length 18, index word length 13, ignore words repeated more than 5 times, maximum mismatches per read 0%, and maximum ambiguity 4.

### Results

Of the nine putative PHAS loci identified, there are six unique miRNA-recognition sequences that were named based on the corresponding miRNAs; 3514, 5770.2, 1510, 1510a.2, 1509, and 1514a.2. It was believed that recognition sequences are bound by miRNAs and induce the production of siRNAs via the ta-siRNA pathway. To test this concept, the miRNA recognition sequences were fused to a transgenic vector. siRNAs made from the mRNA should induce the silencing of complementary sequences.

In order to test if the miRNA recognition sequences identified above would induce the production of siRNAs and lead to gene silencing, two endogenous genes were targeted. Glyma02g43860 encodes a nodulation factor receptor kinase 1 alpha (NFR) and has been shown to control nodule number in soybean (Indrasumunar, et al., Plant Journal 65:39-50 (2011)). Mutants of this gene have reduced nodule numbers. Glyma07g14460 is a putative cytochrome P450 CYP51G1 (P450) and is highly expressed in all tissue types based on RNA-seq data (Libault, Plant J., 63(1):86-99 (2010)). Mutations in CYP51G1 are embryo lethal in arabidopsis (O'Brien, Chantha, et al., 2005).

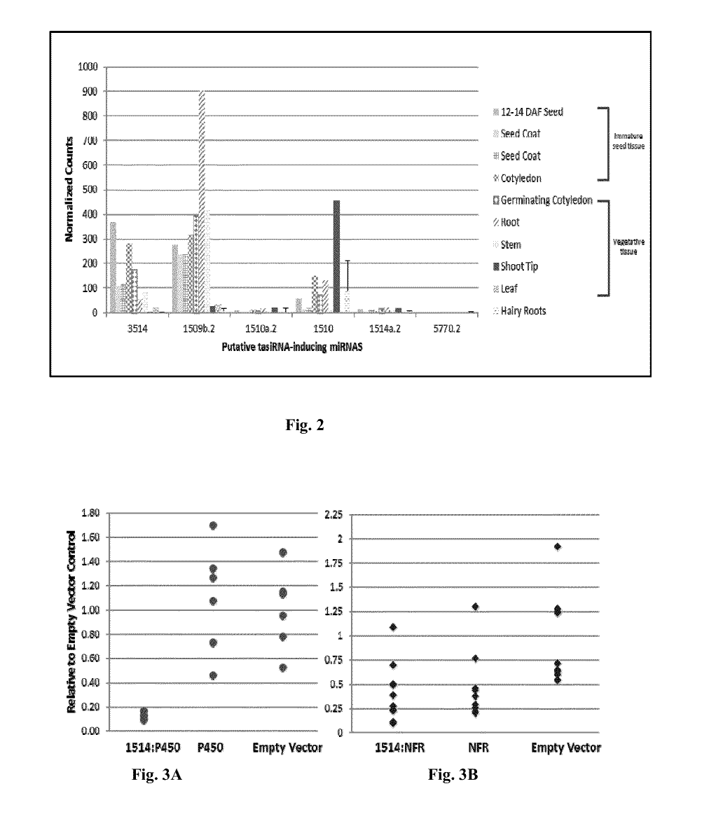

Gene-silencing vectors were made using the 1514a.2 recognition sequence fused to 359 bp and 346 bp portions of NFR and P450, respectively, and introduced into soybean via hairy-root transformation (referred to as 1514:NFR and 1514:P450). Control vectors without the miRNA recognition sequence (referred to as NFR and P450) and empty-vector (wild-type A. rhizogenes) were also used. Hairy root events were grown for two weeks and RNA was extracted for analysis. qRT-PCR results demonstrate that all events transformed with 1514:P450 had less than 20% the amount target transcript compared to the empty vector control (FIG. 3). There was no significant difference in expression between the target control vector (P450) and the no vector control. Individual events from each of the three vectors show a range of NFR expression. Some silencing may be occurring in the 1514:NFR and NFR vectors compared to the empty vector control. This may indicate co-suppression of the NFR target, but may also be due to the relatively low expression of the NFR gene in hairy roots.

Three events from each of the target and control vectors were subjected to small RNA sequencing on an Illumina MiSeq. P450 and NFR events were sequenced in two independent runs. Table 3 summarizes the reads that assembled to silencing cassettes for the respective vectors. In each of the 1514:target events, small RNAs are produced in both the positive and negative strands indicating that dsRNA was produced from these vectors. In the 1514:P450 events there is a bias in counts to the negative strand, whereas in the 1514:NFR events the bias is towards the positive strand. Few counts were observed in the P450 events and these are likely background. One of the NFR events showed the production of a large number of sRNAs, which is probably due to a co-suppression mechanism.

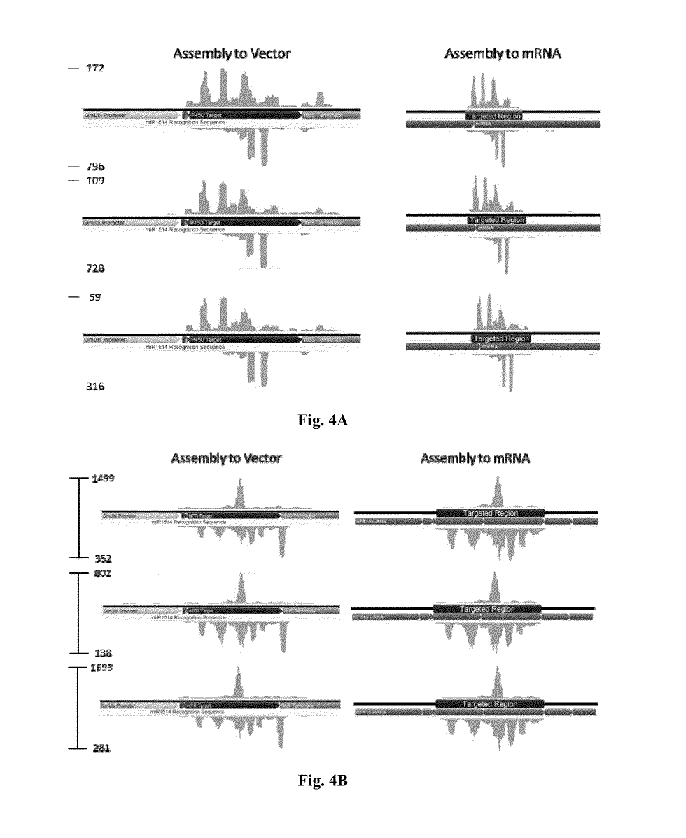

The sRNAs from the 1514:P450 and 1514:NFR events were assembled back to the silencing cassettes and the target mRNA sequences. The coverage graphs show that siRNA production mainly occurs 3′ from the miR1514a.2 recognition sequence and ends in the rbcs terminator (FIGS. 4A and 4B). Within the silencing vectors, the patterns of siRNAs produced are similar. This is likely due to the phased processing of the dsRNA by DCL4 from the initial miRNA recognition sequence. Very few P450 sRNAs assembled to the mRNA sequences beyond the targeted regions while none were observed outside of the NFR target region. This indicates that amplification from the target genes is not occurring. Amplification would result in the production of dsRNAs beyond the targeted region and result in the production of sRNAs outside of the target, in a process known as transitivity. This would indicate that all of the sRNAs are being produced and derived from the transgenic vector.

TABLE 3
Results of small RNA sequencing
Example 3: Fusion of mir1514 Target Site Leads
to Silencing of a Sgfp Transgene

| | | | Normalized Counts in Each | | |
|---|---|---|---|---|---|
| | | | Library Per Million Reads | | |
| Vector | Event | Millions of reads | +strand | −strand | Total |
| 1514:P450 | 1 | 0.365492 | 834 | 2014 | 2848 |
| 1514:P450 | 2 | 0.862042 | 494 | 1593 | 2087 |
| 1514:P450 | 3 | 1.022782 | 243 | 770 | 1013 |
| P450 | 4 | 1.129682 | 3 | 3 | 6 |
| P450 | 5 | 0.617047 | 6 | 3 | 9 |
| P450 | 6 | 0.643904 | 6 | 0 | 6 |
| Total | | 4.640949 | | | |
| 1514:NFR | 7 | 0.530931 | 2375 | 1471 | 3846 |
| 1514:NFR | 8 | 0.802704 | 1186 | 681 | 1867 |
| 1514:NFR | 9 | 0.547316 | 2408 | 1180 | 3588 |
| NFR | 10 | 0.860688 | 4292 | 1404 | 5696 |
| NFR | 11 | 1.096565 | 9 | 1 | 10 |
| NFR | 12 | 0.618872 | 3 | 0 | 3 |
| Total | | 4.457076 | | | |

### Materials and Methods

GFP Protein Quantification

At the time of harvest, 100 mg of root tissue was ground in a 2 mL microcentrifuge tube in phosphate buffer (0.01 M Na2HPO4, 0.15M NaCl, pH 7.5). Phosphate buffer was found to give the lowest background compared to other protein extraction buffers tested. Samples were stored at −80 until all events from an experiment were harvested. GFP was quantified using a Biotek Synergy plate reader according to (Remans, Schenk, et al., 1999). Raw relative fluorescent units (RFU) were normalized by protein concentration. Protein concentration was measured by a Bradford assay (Bradford, 1976).

GFP Imaging

A single three centimeter root tip for each event was removed from the root mass and place on a HRG plate for imaging. All images were taken on an Olympus MVX10 microscope with a GFP filter cube. The imaging software (x) was used and for blue-light images, a 5 ms exposure time was used.

### Results

Given the initial gene-silencing results for the 1514a.2 recognition sequence, sGFP was utilized as a gene-silencing marker to facilitate other gene-silencing experiments. A transgenic soybean line homozygous for sGFP was used (Hernandez-Garcia, Martinelli, et al., 2009). Six gene-silencing vectors were made for sGFP with the putative miRNA recognition sequences, as well as a control vector without recognition sequences as before.

Fifteen randomly selected, individual hairy roots were analyzed under white and blue light. Each root is an individual transgenic event. Gene silencing was observed as hairy roots that do not fluoresce under blue light. Some silencing is observed in the control GFP-Target-Only treatment, but complete silencing is apparent in five of the six miRNA recognition sequences used. More specifically, all of the events from five of the six silencing vectors show a lack of fluorescence under blue light. The no GFP control roots were derived from an isogenic line that lacks the sGFP gene.

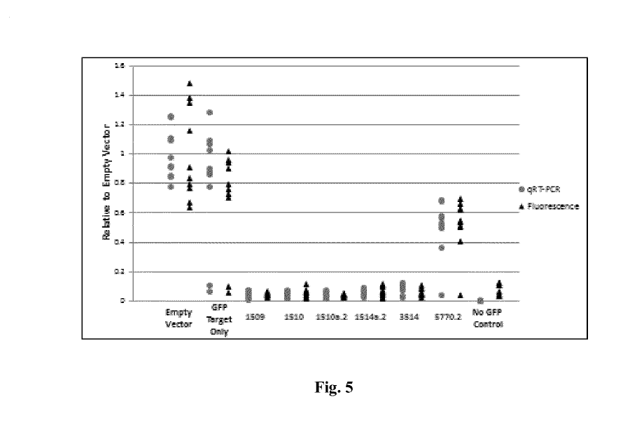

qRT-PCR results from ten randomly selected events show that the five vectors displaying no fluorescence had expression levels less than 20% compared to the empty vector control (FIG. 5). Interestingly, events derived from the 5770.2 targeting vector had approximately 50% the amount of transcript compared to the empty vector control. Silencing presumable caused by co-suppresion was observed in one of the 5770.2 events and in two of the GFP target control events, but was absent in empty vector control. These results were further confirmed by a GFP protein quantification assay from the same events.

Small RNA sequencing of the GFP silenced events reveals the production of sRNAs as for the endogenous genes. In the case of sGFP, there is no apparent strand bias as seen in the endogenous genes. sRNAs do assemble to the sGFP transcript outside of the targeted region suggesting that transitivity is occurring at the sGFP loci as has been previously observed for transgenes.

In these Examples the fusion of the putative soybean miRNAs, including for example miR1514a.2, to portions of two target genes resulted in the silencing of those genes compared to controls as determined by qRT-PCR. Furthermore, sRNA sequencing shows the production of phasiRNA 3′ to the predicted cleavage site. The directionality of phasiRNA production will be useful for avoiding the creation of sRNAs to the promoter, which is known to lead to transcriptional silencing (Mette, et al., Embo Journal 19:5194-5201 (2000)). Upon cleavage of the transcript, it appears that the transitive pathway is not activated, as the sequenced sRNAs do not map outside of the targeted region, indicating this methodology is useful for producing specific species of sRNAs.

In contrast, traditional hairpin vectors (Wesley, et al., Plant Journal 27:581-590 (2001)) are known to induce the transitive pathway (Mlotshwa, et al., Plos One 3:11 (2008)) and so there is a concern of silencing related transcripts when using them, even when the target sequence does not share any homology. Such silencing of unintended genes can have unwanted effects.

### Example 4

### Stable Transformation of Soybean with Silencing Constructs

The 1514:P450, 1514:NFR, 1514:GFP, 1510:GFP, and 1514:GUSPlus silencing vectors were transferred to a biolistic vector and shot into soybean embryogenic cultures to produce stable transgenic lines. Stable events were generated for each of the vectors. Small RNA sequencing of three 1514:P450 and three 1514:GUSPlus events confirms the production of sRNAs in leaf tissue (Table 4).

TABLE 4
Stable Transformation of Soybean with Silencing Constructs

| | | | Millions of | Normalized |
|---|---|---|---|---|
| Vector | Event Number | Counts | reads | Counts |
| 1514:GP | 4 | 9074 | 1.349 | 6726 |
| 1514:GP | 24 | 2649 | 0.714 | 3710 |
| 1514:GP | 12 | 4 | 0.879 | 5 |
| 1514:P450 | 12.2 | 337 | 0.984 | 342 |
| 1514:P450 | 18.2 | 2539 | 1.145 | 2217 |
| 1514:P450 | 20 | 619 | 0.407 | 1521 |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A recombinant nucleic acid construct comprising a tasiRNA-inducing miRNA recognition sequence selected from the group consisting of SEQ ID NOs 5, 1, 2, 3, 4, or 6 and a coding region of a target gene.

2. The recombinant nucleic acid construct of claim 1 further comprising a terminator.

3. The recombinant nucleic acid construct of claim 1 wherein the coding region of the target gene is an exon of the target gene or a fragment thereof.

4. The recombinant nucleic acid construct of any one of claims 1-3 incorporated into an expression vector.

5. The recombinant nucleic acid construct of claim 4 wherein the vector is suitable for transforming the nucleic acid into a plant genome.

6. The recombinant nucleic acid construct of claim 5 wherein the plant genome is a nuclear, mitochondrial, or plastid genome.

7. A transgenic plant transformed with the recombinant nucleic acid construct of claim 1.

8. The transgenic plant of claim 7 wherein the plant is a soybean plant.

9. A method of reducing gene expression in a plant cell comprising transforming the plant cell with a vector that expresses recombinant nucleic acid construct comprising a tasiRNA-inducing miRNA recognition sequence selected from the group consisting of SEQ ID NOs 5, 1, 2, 3, 4, or 6 and a coding region of a target gene,
wherein co-expression of an miRNA that binds to the miRNA target sequence induces production of trans-acting siRNA in the cell.

10. The method of claim 9 wherein the coding region of the target gene comprises 10, 20, 30, 50, 75, 100, 1250, 150, 200, 250, 500, 750, 1000, or more nucleotides.

11. The method of claim 9 wherein the sequence encoding the miRNA is incorporated into the genome of the cell.

12. The method of claim 9 wherein the sequence encoding the miRNA is expressed extra-chromosomally.

13. The method of claim 11 wherein the miRNA sequence is heterologous.

14. The method of claim 11 wherein the miRNA sequence is endogenous.

15. The method of claim 9 wherein reduced expression of the target gene affects a pathway related to the cell's resistance to pathogens, resistance to abiotic stress factors, a biosynthetic pathway, modification of the composition or content of fatty acids, lipids or oils modification of the carbohydrate composition, modification of color or pigmentation, reduction of storage protein content, prevention of stem break, delay of fruit maturation, induction of male sterility, reduction of undesired or toxic plant constituents, delay of senescence symptoms, modification of the lignification or the lignin, or modification of fiber content or quality.

16. A method of reducing gene expression in a plant comprising transforming a plant cell with a vector that expresses a recombinant nucleic acid construct comprising a tasiRNA-inducing miRNA recognition sequence selected from the group consisting of SEQ ID NOs 5, 1, 2, 3, 4, or 6 and a coding region of a target gene,
wherein co-expression of an miRNA that binds to the miRNA target sequence induces production of trans-acting siRNA in the cell.

17. A transgenic plant transformed with a recombinant nucleic acid construct comprising a tasiRNA-inducing miRNA recognition sequence selected from the group consisting of SEQ ID NOs 5, 1, 2, 3, 4, or 6 and a coding region of a target gene.

18. The transgenic plant of claim 17 further comprising a polynucleotide encoding an miRNA that binds under stringent conditions to the miRNA target sequence.

19. The transgenic plant of claim 17 wherein the miRNA sequence at least 90% identical to SEQ ID NO: 12, 7, 8, 9, 10, 11, 13 or a complement thereof.

20. The transgenic plant of claim 17 wherein the plant is a soybean.
